(19) United States
(12) Patent Application Publication
(10) Pub. No.: US 2024/0358822 A1
(43) Pub. Date: Oct. 31, 2024

(54) ALLERGY TREATMENT

(71) Applicant: ENA RESPIRATORY PTY LTD, Sydney (AU)

(72) Inventor: Daphne Tsitoura, Sydney (AU)

(21) Appl. No.: 18/687,841

(22) PCT Filed: Aug. 31, 2022
(86) PCT No.: PCT/AU2022/051062

§ 371 Date: Feb. 29, 2024

(30) Foreign Application Priority Data
Aug. 31, 2021 (AU) 2021902820

(51) Int. Cl. A61K 39/35; A61K 31/23; A61K 39/00; A61K 47/64; A61P 37/08

(52) U.S. Cl. CPC A61K 39/35; A61K 31/23; A61K 47/641; A61P 37/08; A61K 2039/541; A61K 2039/542; A61K 2039/543; A61K 2039/544

(57) ABSTRACT

The invention relates to methods, compounds, compositions and kits for the treatment, attenuation and/or prevention of allergy. In one aspect, the invention relates to the use of the compounds and/or compositions described herein for suppressing or reducing allergen-associated Th2 cell-mediated responses.

### FIELD OF THE INVENTION

The invention relates to methods, compounds, compositions and kits for the treatment, attenuation and/or prevention of allergy. In one aspect, the invention relates to the use of the compounds and/or compositions described herein for suppressing or reducing allergen-associated Th2 cell-mediated responses.

### RELATED APPLICATION

This application claims the benefit of priority to Australian provisional application no. 2021902820 filed 31 Aug. 2021, the entire contents of which is incorporated herein by reference.

### BACKGROUND OF THE INVENTION

Allergic diseases, including allergic rhinoconjunctivitis, allergic asthma, food allergies and atopic dermatitis result from aberrant Th2 immune reactivity against common environmental allergens, such as pollen, house dust mite, animal dander, moulds and food. Th2 cells, through the secretion of cytokines (i.e. IL-4, IL-5, IL-9, IL-13), drive two critical elements responsible for the manifestation of allergic symptoms: a) increased synthesis of allergen-specific IgE, which mediates type I hypersensitivity reactions, and b) differentiation, survival and chemotaxis of proinflammatory cells, namely mast cells, basophils and eosinophils that orchestrate the development of chronic inflammation.

The prevalence of allergic diseases is increasing worldwide, and as a consequence there is an increased financial burden on the health care system. Existing therapies alleviate symptomatology, but do not alter the disease process. The only therapeutic modality that has been associated with the development of allergen desensitisation, immune tolerance and potentially disease remission through modification of the underlying disease pathophysiology, is allergen specific immunotherapy.

Allergen immunotherapy involves regular administration of gradually increasing doses of allergen extracts over long periods of time, usually 3-5 years. Despite promising efficacy, current allergen immunotherapies are associated with several limitations, such as inconvenient dosing regimens, long treatment periods, poor treatment adherence, variable response to treatment, poor local tolerability and may be associated with potentially serious side effects (e.g. life-threatening systemic allergic reactions). These limitations restrict broad application to patients suffering from allergy and highlight the need for novel, alternative forms of allergen-specific or non-specific immunotherapy that modulate the aberrant, pathogenic Th2 responses to allergens.

Given the above, there remains a need for improved therapies for the treatment and/or prevention of allergy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In an aspect of the invention, there is provided a method of treating, attenuating or preventing an allergic immune response in a subject, comprising administering a therapeutically effective amount of a TLR2 moiety comprising, consisting essentially of, or consisting of a TLR2 agonist and a polar polypeptide,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof, thereby treating, attenuating or preventing an allergic immune response in the subject.

In any aspect of the invention, the lipid moiety is palmitolyl.

In any aspect of the invention, the TLR2 agonist is a lipopeptide. The lipopeptide may be any compound comprising a fatty acid moiety and an amino acid moiety.

The lipopeptide may comprise one or more fatty acid moieties which may be the same or different. In an embodiment, the lipopeptide comprises one, two or three fatty acid residues.

The lipopeptide may comprise one or more amino acid moieties, which may be the same or different and optionally are linked together though a peptide bond.

The fatty acid moiety and amino acid moiety may be linked via a linker. The linker may be an alkyl group substituted with functional groups capable of forming bonds to a fatty acid and an amino acid. In an embodiment, the linker and each fatty acid moiety are bonded through an oxygen atom, such as in the form of a carboxy linkage. In an embodiment, the linker and the amino acid are bonded via the amino acid side-chain, for example, when the amino acid moiety comprises a cysteine residue, the linker may bond directly with the cysteine sulphur atom.

In an embodiment, the lipopeptide may be selected from the group consisting of Pam2Cys, Pam3Cys, Ste2Cys, Lau2Cys, and Oct2Cys. Preferably, the lipopeptide is Pam2Cys. In another embodiment, the lipopeptide may be diacylated with the lipid moiety.

In any aspect of the invention, the polar polypeptide is a branched polypeptide. In another aspect, the polar polypeptide may be a hyper-branched polypeptide. In one embodiment, the branched or hyper-branched polypeptide is any one described herein. In one embodiment, the branched or hyper-branched polypeptide further comprises PEG.

In any aspect, the branched or hyper-branched polypeptide may comprise one or more amino acids selected from the group consisting of aspartic acid (Asp, D) glutamic acid (Glu, E), histidine (His, H), lysine (Lys, K), arginine (Arg, R), asparagine (Asn, N), glutamine (Gln, Q), serine (Ser, S), threonine (Thr, T) and tyrosine (Tyr, Y).

In any aspect, the branched polypeptide comprises a dendrite structure terminating in a plurality of up to four amino acids. In any aspect, the hyper-branched polypeptide is a polymer that comprises a dendrite structure terminating in a plurality of five, six, seven, eight or more amino acids.

In any aspect of the invention, the polar polypeptide may comprise, consist essentially of, or consist of a positively or negatively charged group. Preferably, the charged group is any branched or hyper-branched polypeptide described herein. Preferably, the positively charged group comprises positively charged amino acids capable of forming a branched polypeptide or hyper-branched polypeptide, such as arginine or lysine residues. Preferably, the negatively charged group comprises negatively charged amino acids capable of forming a branched polypeptide or hyper-branched polypeptide, such as glutamate or aspartate. The charged amino acids may be terminal, preferably N-terminal.

In any aspect of the invention, polar polypeptides for use in any of the methods described herein may include “R4”, a branched tetra arginine peptide; “R8”, a hyper-branched arginine peptide “H4”, a branched tetra histidine peptide; “H8”, a peptide containing hyper-branched histidine residues; “K4”, a peptide containing branched lysine residues; “K8”, a peptide containing hyper-branched lysine residues; “E4” a branched peptide containing glutamate residues, “E8” a hyper-branched peptide containing glutamate residues, and “D8” a hyper-branched peptide containing aspartate residues as described herein. In any embodiment of the invention, including those described above, the polar polypeptide is preferably R4.

Preferably, the TLR2 agonist and the polar polypeptide are conjugated or linked. In an embodiment, the TLR2 agonist and the polar polypeptide are linked by at least one serine residue, preferably two serine residues.

In any aspect of the invention, the TLR2 moiety comprising, consisting essentially of, or consisting of a TLR2 agonist and a polar polypeptide is Pam2CysR4 (alternatively denoted herein as INNA-001), and having the structure:

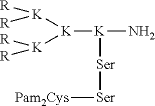

In accordance with the above, in an aspect, there is therefore provided a method of treating, attenuating or preventing an allergic immune response in a subject comprising administering a therapeutically effective amount of Pam2CysR4, thereby treating, attenuating or preventing the allergic immune response in the subject.

In any aspect of the invention, the TLR2 moiety may not comprise polyethylene glycol (PEG).

In any aspect of the invention, the allergic immune response is a hypersensitivity reaction initiated by immunologic mechanisms and triggered by exposure to a defined stimulus or any allergen described herein at a dose tolerated by a subject.

In any aspect of the invention, the allergic immune response is associated with an allergy selected from:
microbe allergy;
pollen allergy;
animal allergy;
mite or insect allergy;
mould allergy;
plant allergy;
food allergy;
aeroallergens;
drug allergy; and
hymenoptera venom allergy.

In a preferred aspect, the allergic immune response is associated with pollen allergy or animal allergy.

In an aspect of the invention, there is provided a method of treating, attenuating or preventing an allergic immune response in a subject, comprising administering a therapeutically effective amount of (i) a TLR2 moiety comprising, consisting essentially of, or consisting of a TLR2 agonist and a polar polypeptide; and (ii) and an allergen,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof,

thereby treating, attenuating or preventing an allergic immune response in the subject.

In an aspect of the invention, there is provided a method of treating, attenuating or preventing an allergic immune response in a subject comprising administering a therapeutically effective amount of a (i) Pam2CysR4; and (ii) an allergen, thereby treating, attenuating or preventing the allergic immune response in the subject.

In any aspect of the invention, the allergen to be administered with the TLR2 moiety described herein or with Pam2CysR4 may be selected from any type of allergen described herein, including, but not limited to:
microbe allergens;
aeroallergens;
pollen allergens;
animal allergens;
mite or insect allergens;
mould allergens;
plant allergens;
food allergens;
drug allergens; and
hymenoptera venom allergens.

In any aspect, the allergen may be an allergen derivative including but not limited to hypo-allergens, recombinant allergens, peptides from allergens or a derivative of any allergen described herein.

In any aspect of the invention, the allergen to be used in accordance with any method described herein is a plant or animal allergen. In a preferred embodiment, the allergen is Timothy grass pollen allergen (Phleum pratense) or the cat allergen Fel d 1.

In any aspect of the invention, the subject to be treated for an allergic immune response exhibits one or more symptoms associated with an allergic response including sneezing; itchy, runny and/or blocked nose; itchy, red and/or watering eyes; wheezing and/or chest tightness; reduced shortness of breath and/or coughing; swelling (eg of lips); nausea; vomiting and/or diarrhoea; rash and/or itchiness.

In any aspect of the invention, the subject receiving treatment for an allergic immune response exhibits one or more symptoms associated with anaphylaxis including swelling of throat, difficulty in breathing, light headedness, cyanosis of lips and/or skin and collapsing and/or losing consciousness. In a preferred embodiment, the subject has been exposed to Timothy grass pollen allergen (Phleum pratense) or the cat allergen Fel d 1.

In any aspect of the invention, the subject receiving treatment for an allergic immune response has received, or is receiving a treatment in the form of an allergen. Preferably, the subject has received, or is receiving a treatment in the form of Timothy grass pollen allergen (Phleum pratense) or the cat allergen Fel d 1. In this embodiment, the subject may exhibit one or more of those symptoms described above. In another embodiment, the severity of the one or more symptoms of the subject that has received, or is receiving a treatment in the form of an allergen may be reduced relative to a subject not receiving treatment.

In any aspect of the invention, the subject that has received or is receiving treatment in the form of the allergen is receiving the treatment via the respiratory system (inhalation or intranasally), topically, orally, intravenously, intramuscularly, cutaneously or via any other administration route described herein or known in the art.

In any aspect of the invention, the allergy to be treated, attenuated and/or prevented is not viral mediated exacerbation of a respiratory condition. In another embodiment, the allergy to be treated, attenuated and/or prevented is not viral mediated exacerbation of asthma. In this context, the virus may be rhinovirus.

In an aspect of the invention, there is provided a method of desensitising and/or tolerising a subject to an allergen comprising administering a therapeutically effective amount of a TLR2 moiety comprising, consisting essentially of, or consisting of a TLR2 agonist and a polar polypeptide,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof,

thereby desensitising and/or tolerising the subject to an allergen.

In an aspect of the invention, there is provided a method of desensitising and/or tolerising a subject to an allergen comprising administering a therapeutically effective amount of a TLR2 moiety comprising, consisting essentially of, or consisting of a (i) TLR2 agonist and a polar polypeptide; and (ii) an allergen,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof;

thereby desensitising and/or tolerising the subject to an allergen.

In any aspect of the invention, the allergen is any allergen described herein. In another embodiment, the TLR2 moiety is any TLR2 moiety described herein. Preferably, the TLR2 moiety is Pam2CysR4.

In an aspect of the invention, there is provided a method of desensitising and/or tolerising a subject to an allergen comprising the steps of:
identifying a subject that is allergic to an allergen; and
administering a therapeutically effective amount of a TLR2 moiety comprising a TLR2 agonist and a polar polypeptide,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof;

thereby desensitising and/or tolerising the subject to an allergen.

In an aspect, the method may further comprise administration of an allergen. The allergen may be any allergen described herein.

In any aspect where desensitisation and/or tolerisation of a subject is contemplated, the TLR2 moiety and/or allergen treatment disclosed herein is continued for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 18 months, at least about 24 months, at least about 3 years, at least about 5 years, or at least about 10 years, or any period between.

In an aspect of the invention, there is provided a method of increasing levels of regulatory T and/or regulatory B cells in a subject, comprising administering a therapeutically effective amount of any TLR2 moiety described herein to the subject, thereby increasing levels of regulatory T and/or regulatory B cells in the subject.

In an aspect of the invention, there is provided a method of increasing levels of monocytes in a subject comprising administering a therapeutically effective amount of any TLR2 moiety described herein to the subject, thereby increasing levels of monocytes in the subject. Preferably, the monocytes are non-classical monocytes. In another preferable aspect, the monocytes are interleukin 10 (IL-10) producing monocytes.

In an aspect of the invention, there is provided a method of treating, attenuating or preventing anaphylaxis in a subject comprising administering a therapeutically effective amount of any TLR2 moiety described herein to the subject, thereby treating, attenuating or preventing anaphylaxis in the subject.

In an aspect of the invention, there is provided a method of increasing survival in a subject comprising administering a therapeutically effective amount of any TLR2 moiety described herein to the subject, thereby increasing survival in the subject.

In an aspect of the invention, there is provided a method of suppressing or reducing levels of IL-4, IL-5 and/or IL-13 in a subject comprising administering a therapeutically effective amount of any TLR2 moiety described herein to the subject, thereby suppressing or reducing levels of IL-4, IL-5 and/or IL-13 in the subject.

In an aspect of the invention, there is provided a method of treating, alleviating or preventing a Th2 immune response in a subject comprising administering a therapeutically effective amount of any TLR2 moiety described herein to the subject, thereby treating, alleviating or preventing a Th2 immune response in the subject. In one aspect, the treatment, alleviation or prevention of a Th2 immune response is determined by the assessment of levels of IL-4, IL-5 and/or IL-13 by routine means known in the art or described herein in the Examples. In another aspect, the treatment, alleviation or prevention of a Th2 immune response is determined by the assessment of Th2 cell proliferation using routine means known in the art or described herein in the Examples.

In an aspect of the invention, there is provided a method of increasing levels of IL-10 in a subject comprising administering a therapeutically effective amount of any TLR2 moiety described herein to the subject, thereby increasing levels of IL-10 in the subject. In another aspect, the method comprises increasing levels of IL-27 is a subject.

In any aspect of the invention, the administration of any TLR2 moiety described herein is not associated with a substantial Th1 response. In another embodiment, the administration of any TLR2 moiety described herein is not associated with substantial changes to levels of interferon gamma (IFN gamma). In this aspect, this does not mean that levels of IFN gamma or a Th1 response is not clinically or biochemically detectable. A substantial Th1 response or a substantial change to levels of IFN gamma may be determined by any routine means in the art including that described herein in the Examples.

In any aspect of the invention, the TLR2 moiety is a lipid moiety selected from the group consisting of palmitolyl, myristroyl, steareoyl, lauroyl, decanoyl and combinations thereof. Preferably, the lipid moiety is palmitolyl.

In an aspect of the invention, there is provided a composition comprising a TLR2 moiety and an allergen,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof.

In this aspect, the allergen may be any allergen described herein.

In any aspect of the invention, the (i) TLR2 moiety and the (ii) allergen may be administered at the same time. Alternatively, they may be administered sequentially. For instance, the allergen may be administered prior to the TLR2 moiety or the TLR2 moiety may be administered prior to the allergen. Alternatively, treatment with the allergen and/or TLR2 moiety may be staggered. In another aspect, the (i) TLR2 moiety and the (ii) allergen may be administered at least once. In another embodiment, the (i) TLR2 moiety and the (ii) allergen may be administered one, two, three, four, five, six, seven, eight, nine or ten times, or up to twenty, thirty, forty, fifty, a hundred or more times in accordance with any of the administration regimes outlined above. In another embodiment, the TLR2 moiety and the (ii) allergen may be administered more than once.

In any aspect of the invention, the (i) TLR2 moiety and/or (ii) allergen may be administered in a composition. In any aspect of the invention, the (i) TLR2 moiety and/or (ii) allergen may be administered in the same composition or in separate compositions. Typically, the composition further comprises a pharmaceutically acceptable carrier, diluent or excipient. The composition may be formulated for systemic, intravenous, intralymphatic, intramuscular, subcutaneous, intradermal, oral, topical, intranasal, aerozolised, epidermal or sublingual administration to the subject. Preferably, the composition is formulated for oral or sublingual administration.

In any aspect of the invention, the TLR2 moiety is administered in the form of a composition, which may be free of compounds that are agonists of other TLRs. Preferably, the only TLR agonist present in the composition is an agonist of TLR2 homodimers or heterodimers. Preferably, the composition comprising the TLR2 moiety only contains one TLR2 agonist.

In any aspect of the invention, the composition comprises any TLR2 moiety described herein, and a pharmaceutically acceptable carrier, diluent or excipient.

In an aspect of the invention, there is further provided a composition comprising (i) any TLR2 moiety described herein, (ii) an allergen, and a (iii) pharmaceutically acceptable carrier, diluent or excipient.

In an aspect of the invention, there is further provided use of a TLR2 moiety comprising, consisting essentially of or consisting of a TLR2 agonist and a polar polypeptide in the preparation of a medicament for treating, attenuating or preventing an allergic immune response in a subject,

wherein the TLR2 agonist comprises, consists essentially of or consists of a lipopeptide or lipid moiety,

wherein the lipid moiety is selected from the group consisting of palmitolyl, myristroyl, steareoyl, lauroyl, decanoyl and combinations thereof.

In any aspect, the medicament may further comprise any allergen described herein. In a preferred embodiment, the TLR2 moiety is Pam2CysR4.

In an aspect of the invention, there is provided use of any TLR2 moiety described herein in the manufacture of a first medicament, and/or any allergen described herein in the preparation of a second medicament, wherein the first and/or second medicaments are for:
treating, attenuating or preventing an allergic immune response in a subject,
inducing desensitisation and/or tolerance to an allergen in a subject,
increasing levels of regulatory T and/or regulatory B cells and/or levels of IL-10 producing non-classical monocytes, antigen presenting cells or other immune cells associated with an allergic immune response in a subject,
treating, attenuating or preventing anaphylaxis associated with an allergic immune response;
suppressing or reducing levels of IL-4, IL-5 and/or IL-13 associated with an allergic immune response;
treating, alleviating or preventing a Th2 immune response;
increasing levels of IL-10 associated with an allergic immune response; or
increasing survival of a subject.

Alternatively, the first and second medicaments are for any other method or use of the invention as described herein.

In an aspect of the invention, there is further provided use of any TLR2 moiety described herein for treating, attenuating or preventing an allergic immune response in a subject. The present invention further provides (i) any TLR2 moiety described herein; and (ii) any allergen described herein for treating, attenuating or preventing an allergic immune response in a subject.

In an aspect of the invention, there is further provided any TLR2 moiety described herein for use in treating, attenuating or preventing an allergic immune response in a subject. The present invention further provides (i) any TLR2 moiety described herein; and (ii) any allergen described herein for use in treating, attenuating or preventing an allergic immune response in a subject.

Alternatively, the (i) TLR2 moiety and/or (ii) allergen is for use in any other method or use of the invention as described herein.

In an aspect of the invention, there is further provided use of any TLR2 moiety described herein in the manufacture of a medicament for:
treating, attenuating or preventing an allergic immune response in a subject who has received, or is receiving treatment in the form of an allergen,
treating, attenuating or preventing an allergic immune response in a subject who is sensitised to one or more allergens described herein with one or more symptoms described herein,
inducing desensitisation and/or tolerance to an allergen in a subject who has received, or is receiving treatment in the form of an allergen,
treating, attenuating or preventing anaphylaxis associated with an allergic immune response in a subject who has received, or is receiving treatment in the form of an allergen,
increasing survival associated with an allergic immune response in a subject who has received, or is receiving treatment in the form of an allergen, or
suppressing or reducing levels of IL-4, IL-5 and/or IL-13 or treating, alleviating or preventing a Th2 immune response in a subject who has received, or is receiving an treatment in the form of an allergen.

In any aspect of the invention, the TLR2 agonist is not Pam3Cys.

In any aspect of the invention, the amount of any TLR2 moiety described herein administered may be in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose.

In any aspect of the invention, the amount of TLR2 moiety to be administered may be in the range of about 0.1, 0.2, 0.3, 0.4, 0.5, 0.6, 0.7, 0.8, 0.9, 1.0, 5, 10, 15, 20, 25, 30, 35, 40, 45, 50, 55, 60, 65, 70, 75, 80, 85, 90, 95 or 100 μg/kg or more.

In any aspect of the invention, the amount of any allergen described herein may be in the range of from about 0.1 to about 20 ng, about 0.1 to about 10 ng, about 0.1 to about 5 ng, about 1 to about 5 ng, about 2 to about 5 ng, about 7.5 to about 12.5 ng, about 0.1 to about 30 ng, about 0.1 to about 100 ng, about 0.1 to about 500 ng, about 0.1 to about 1000 ng, about 0.1 to about 2000 ng, about 0.1 to about 100 μg, about 0.1 to about 250 μg, about 0.1 to about 500 μg, about 0.1 to about 750 μg, about 0.1 to about 1000 μg, about 0.1 to about 0.25 mg, about 0.1 to about 0.5 mg, about 0.1 to about 0.75 mg, about 0.1 to about 1.0 mg or about 0.1 to about 1.25 mg or about 0.1 to about 1.5 mg. For example, dosages can be about 0.1, about 0.3, about 1, about 2, about 3, about 5 or about 10 ng, or, about 0.3, about 1, about 2, about 3, or about 5 ng. In another aspect of the invention, the amount of any allergen described herein may be in the range of about 0.5 to 5 ng, preferably in the range of about 1 to 3 ng, more preferably 1.23-2.46 ng. In another embodiment, the amount of any allergen described herein may be in the range of about 0.1 ng to 1 mg.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. INNA-001 reduces allergen-driven Th2 cytokine production and proliferation. PBMC from grass pollen allergic patients (N=8) were cultured for 6 days with or without allergen (Phlp at 10 μg/mL) and with or without INNA-001 at 3 μg/mL (INNA-001 or control TLR2 agonists where pre-mixed with the allergen prior to addition to the cell cultures). At day 6, supernatants were harvested and the levels of Th2 cytokines (IL-4, IL-5 and IL-13) was analysed via a Luminex MagPix. Cells were pulsed with ³H-Thymidine for the final 18 hours of culture before harvesting and cell proliferation was measured. All data shown are mean±SEM. Friedman's test (*p<0.05, **p<0.01, ***p<0.001).

FIG. 2. INNA-001 reduces allergen-driven Th2 cytokine production and proliferation. (A) PBMC from grass pollen allergic patients (N=8) were cultured for 6 days with or without allergen (Phlp at 10 μg/mL) and with or without a range of concentrations (0.03 to 3 μg/ml) of INNA-001 (A, B, C) or INNA-003 (D, E, F). After 6 days, supernatants were harvested and the levels of cytokines were analysed via a Luminex MagPix. Friedman's test (*p<0.05, **p<0.01, ***p<0.001). (B) PBMC from Timothy grass pollen allergic patients (N=3) were cultured for 6 days with or without allergen (Phlp at 10 μg/mL) and with or without a range of concentrations (3 to 10 μg/mL) of INNA-006 (NNA-006 was pre-mixed with the allergen prior to addition to the cell cultures). After 6 days, supernatants were harvested, and the levels of cytokines were analysed via a Luminex MagPix. All data shown are mean±SEM. Statistical analysis was performed using Friedman's test (*p<0.05, **p<0.01, ***p<0.001).

FIG. 3. Treatment with INNA-001 suppresses the expansion of pro-allergic Th2 cell phenotypes in response to allergen stimulation. PBMC from grass pollen allergic patients (N=12) were cultured for 6 days with or without allergen (Phlp at 10 μg/mL) and with or without a range of concentrations of INNA-001. After 6 days, the cells were harvested, washed and activated with phorbol 12-myristate 13-acetate (25 ng/mL) and ionomycin (1 μg/mL) for 5 hours. Brefeldin A (10 μg/mL) was added for the final 4 hours of stimulation. Subsequently the cells were washed and stained for anti-human CD4, CD27, CRTH2, CD49d, CD161, IL-13 and IL-4. Analysis was performed by Flow Cytometry. Proliferation of cells was measured using CellTrace violet. All data shown are mean±SEM. Graphs represent normalised changes from baseline (untreated). Statistical analysis was performed using ANOVA with Dunn's multiple comparisons test for within-group comparisons (*p<0.05, **p<0.01, ***p<0.001 for the +Phlp group).

FIG. 4. INNA-001 reduces allergen-driven Th2 cytokine production and proliferation. PBMC from grass pollen allergic patients (N=3) were cultured for 6 days with or without allergen (Phlp at 10 μg/mL) and with or without a range of concentrations of INNA-001, INNA-003, INNA-006 or Pam2CSK4 (0.3, 3 and 10 g/ml). After 6 days, the cells were harvested, washed and activated with phorbol 12-myristate 13-acetate (25 ng/mL) and ionomycin (1 μg/mL) for 5 hours. Brefeldin A (10 μg/mL) was added for the final 4 hours of stimulation. Subsequently the cells were washed and stained for anti-human CD4, CD45RO+CD27, CRTH2 and IL-13. Analysis was performed by Flow Cytometry. All data shown are mean±SEM. Graphs represent normalised changes from baseline (untreated). Statistical analysis was performed using ANOVA with Dunn's multiple comparisons test for within-group comparisons (*p<0.05, **p<0.01, ***p<0.001 for the +Phlp group).

FIG. 5. INNA-001 selectively suppresses the expansion of pro-inflammatory Tfh cells in response to allergen stimulation. PBMC from grass pollen allergic patients (N=3) were cultured for 6 days with or without allergen (Phlp at 10 μg/mL) and with or without a range of concentrations of INNA-001. After 6 days, the cells were harvested, washed and activated with phorbol 12-myristate 13-acetate (25 ng/mL) and ionomycin (1 μg/mL) for 5 hours. Brefeldin A (10 μg/mL) was added for the final 4 hours of stimulation. Subsequently the cells were washed and stained for anti-human CD4, CXCR5, PD-1 and IL-4 or IL-21. Analysis was performed by Flow Cytometry. Proliferation of cells was measured using CellTrace violet. All data shown are mean±SEM. Graphs represent normalised changes from baseline (untreated). Statistical analysis was performed using ANOVA with Dunn's multiple comparisons test for within-group comparisons (*p<0.05, **p<0.01, ***p<0.001 for the +Phlp group).

FIG. 6. INNA-001 does not upregulate the production of Thi-type cytokines by human PBMC. PBMC from grass pollen allergic patients (N=3) were cultured for 6 days with or without allergen (Phlp at 10 μg/mL), in the presence or absence of INNA-001, INNA-003 or INNA-006 (at 3 and 10 μg/mL). At day 6, supernatants were harvested and the levels of Th1 cytokines (IFN-gamma and IL-12p70) were analysed via a Luminex MagPix.

FIG. 7. INNA-001 promotes the induction of IL-10 and IL-27 by human PBMC. PBMC from grass pollen allergic patients (N=3) were cultured for 6 days with or without allergen (Phlp at 10 μg/mL), in the presence or absence of INNA-001, INNA-003 or INNA-006 (at 3 and 10 μg/mL). At day 6, supernatants were harvested and the levels of IL-27 and IL-10 were analysed via a Luminex MagPix.

FIG. 8. PBMC treatment with INNA-001 upregulates the production of IL-6. PBMC from grass pollen allergic patients (N=3) were cultured for 6 days with or without allergen (Phlp at 10 μg/mL), in the presence or absence of INNA-001, INNA-003 or INNA-006 (at 3 and 10 μg/mL). At day 6, supernatants were harvested and the levels of IL-6 were analysed via a Luminex MagPix. All data shown are mean±SEM. Statistical analysis was performed using ANOVA with Dunn's multiple comparisons test for within-group comparisons (*p<0.05, **p<0.01, ***p<0.001 for the +Phlp group).

FIG. 9. Treatment of PBMC with INNA-001 or INNA-006 triggers rapid IL-10 and IL-6 induction. PBMC from grass pollen allergic patients (N=3) were cultured for 24 hours with or without a range of allergen concentrations (Phlp at 0.1, 1 or 10 mg/mL), in the presence or absence of a range of INNA-001, INNA-006 or Pam2CSK4 concentrations (from 0.01 to 10 μg/mL). The culture supernatants were harvested and the levels of IL-10 (A) and IL-6 (B) were measured by ELISA. All data shown are mean±SEM.

FIG. 10. Treatment of PBMC, from grass allergic subjects, with INNA-001, but not INNA-006, triggers rapid and sustained IL-10 induction. PBMC from grass pollen allergic patients (N=3) were cultured for 24 hours, 72 hours or 6 days, with or without allergen (Phlp 10 mg/mL), in the presence or absence of a range of INNA-001, INNA-006 or Pam3CSK4 concentrations (0.3, 3, 10 and 30 μg/mL). The culture supernatants were harvested and the levels of IL-10 were measured by ELISA. All data shown are mean±SEM.

FIG. 11. INNA-001 induces dose-dependent upregulation of IL-10 production in PBMC from cat allergic patients. PBMC from cat allergic patients (N=3) were cultured for 24 hours, 72 hours or 6 days, with or without allergen (Feld 1 0.1 or 1 g/mL), in the presence or absence of a range of INNA-001, INNA-006 or Pam2CSK4 concentrations (0.3, 3, 10 and 30 μg/mL). The culture supernatants were harvested and the levels of IL-10 were measured by Elisa.

FIG. 12. PBMC treatment with INNA-001, in the presence of allergen, selectively expands IL-10 producing, CD14low/CD16+ non-classical monocytes. PBMC from grass pollen allergic patients were cultured for 24 hours with or without allergen (Phlp at 10 μg/mL) and with or without a range of concentrations of INNA-001 or Pam2CSK4 (0.3, 3 and 10 μg/mL). At 24 hours the cells were harvested, washed and stained for anti-human CD14, CD16 and IL-10. Analysis was performed by Flow Cytometry. A) Flow Cytometry representative plots (N=3) for percentage of IL-10-producing subpopulation of non-classical (CD14^{low} CD16⁺) monocytes after treatment with 0.3 mg/mL of TLR2 agonists (INNA-001 or Pam2CSK4) in the presence of Phlp. B) Quantification (N=12) of IL-10 producing non-classical (CD14^{low} CD16⁺) and C) Quantification (N=12) of IL-10 producing classical (CD14^{high}CD16⁻) monocytes. All data shown are mean±SEM. Graphs represent changes from baseline (untreated). Statistical analysis was performed using ANOVA with Dunn's multiple comparisons test for within-group comparisons. Mann-Whitney U test was performed for between-group comparisons. Statistical significance in the +Phlp group: *p<0.05, **p<0.01, ***p<0.001.

FIG. 13. PBMC treatment with INNA-001, INNA-003 and INNA-006, in the presence of allergen, induces IL-10 producing, CD14^{low}/CD16⁺ non-classical monocytes. PBMC from grass pollen allergic patients (N=3) were cultured for 24 hours with or without allergen (Phlp at 10 μg/mL) and with or without a range of concentrations of INNA-001, INNA-003 or INNA-006 (0.3, 3, 10 and 30 μg/ml). At 24 hours the cells were harvested, washed and stained for anti-human CD14, CD16 and IL-10. Analysis was performed by Flow Cytometry. All data shown are mean±SEM.

FIG. 14. INNA-001 promotes the expansion of FoxP3+T regulatory cells. PBMC from grass pollen allergic patients were cultured for 6 days with or without allergen (Phlp at 10 μg/mL) and with or without a range of concentrations of INNA-001 or Pam2CSK4 (0.3, 3 and 101 g/ml). At day 6 the cells were harvested, washed and stained for anti-human CD4, CD25, CD127 and Foxp3. Analysis was performed by Flow Cytometry. All data shown are mean±SEM. Graphs represent changes from baseline (untreated). Statistical analysis was performed using ANOVA with Dunn's multiple comparisons test for within-group comparisons. Mann-Whitney U test was performed for between-group comparisons. Statistical significance in the +Phlp group: *p<0.05, **p<0.01, ***p<0.001.

FIG. 15. INNA-001 promotes the expansion of naïve and memory subpopulation FoxP3+CD4+T regulatory cells. PBMC from grass pollen allergic patients were cultured for 6 days with or without allergen (Phlp at 10 μg/mL) and with or without a range of concentrations of INNA-001 or Pam2CSK4 (0.3, 3 and 10 μg/ml). At day 6 the cells were harvested, washed and stained for anti-human CD4, CD25, CD127, Foxp3 and CD45RO. Analysis was performed by Flow Cytometry. All data shown are mean±SEM. Graphs represent changes from baseline (untreated) (A) naive FoxP3⁺CD25⁺CD127⁻CD4⁺CD45RO⁻ cells, and (B) memory FoxP3⁺CD25⁺CD127⁻CD4⁺CD45RO⁺ cells. Statistical analysis was performed using ANOVA with Dunn's multiple comparisons test for within-group comparisons. Mann-Whitney U test was performed for between-group comparisons. Statistical significance in the +Phlp group: *p<0.05, **p<0.01, ***p<0.001.

FIG. 16. INNA-001, in the presence of allergen, promotes more efficiently than INNA-003 or INNA-006 the expansion of memory FoxP3⁺CD4⁺ T regulatory cells. PBMC from grass pollen allergic patients (N=3 for INNA=003 & INNA-006, N=7 for INNA-001 & Pam2CSK4) were cultured for 6 days with or without allergen (Phlp at 10 μg/mL) and with or without a range of concentrations of INNA-001, INNA-003, INNA-006 or Pam2CSK4 (0.3, 3 and 10 μg/mL). At day 6 the cells were harvested, washed and stained for anti-human CD4, CD25, CD127, Foxp3 and CD45RO. Analysis was performed by Flow Cytometry. All data shown are mean±SEM.

FIG. 17. INNA-001, in the presence of allergen, expands different phenotypic subsets of Foxp3⁺ T regulatory cells. PBMC from grass pollen allergic patients were cultured for 6 days with allergen (Phlp at 10 μg/mL) and with or without INNA-001 or Pam2CSK4 (3 μg/mL). At 6 days the cells were harvested, washed and stained for anti-human Foxp3, CD4, CD25, CD45RO, CD127, CTLA4 and SABT1. Analysis was performed by Flow Cytometry. The data were further analysed using unbiased FlowSOM technique. Foxp3⁺ T regulatory cell metacluster identification following treatment with INNA-001 A) in the presence of Phlp (N=3) or B) absence of Phlp (N=3). C) Heatmap display of the marker expression profile of the identified metaclusters (N=3). D) Quantification of population abundance of the metaclusters 3, 5 and 9 following INNA-001 and Pam2CSK4 treatment in the presence of Phlp (N=3).

FIG. 18. INNA-001 induces expansion of regulatory B cells. CD19⁺ B cells were isolated from PBMC of grass pollen allergic patients (N=3) and were cultured for 72 hours with or without a range of INNA-001 concentrations (0.03, 0.3 and 3 g/ml) in the presence or absence of allergen (Phlp at 10 μg/mL). At 72 hours the cells were harvested, washed and stained for anti-human CD5, CD27, CD24, CD38 and IL-10. Analysis was performed by Flow Cytometry. All data shown are mean±SEM.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

Allergic diseases, including allergic rhinoconjunctivitis, allergic asthma, food allergies and atopic dermatitis result from aberrant Th2-type immune reactivity against common environmental allergens. Existing therapies alleviate symptomatology, but do not alter the underlying disease process and therefore do not offer long-lasting benefits. The only therapeutic modality that has been associated with the development of allergen desensitisation, immune tolerance and potentially disease remission, through modification of the underlying disease pathophysiology, is allergen specific immunotherapy.

Although efficacious when used appropriately, allergen immunotherapy, when used as a single therapeutic approach, has several limitations, such as inconvenient dosing regimens, long treatment periods, poor treatment adherence, variable response to treatment, poor local tolerability and may be associated with potentially serious side effects (e.g. life-threatening systemic allergic reactions).

Given the limitations associated with current allergy immunotherapy approaches, the inventors set out to determine whether the administration of an immunopotentiator, alone or in combination allergen, can effectively treat, attenuate or prevent an allergic response in allergic subjects. In particular, the inventors sought to determine whether selective stimulation of the Toll-like receptor 2 (TLR2) immune cascade can result in modulation of the phenotype of human allergen-specific Th2 cell-mediated immune responses and enhancement of counter-regulatory immune mechanisms. It is herein established for the first time that the compounds and compositions described herein have utility for allergen immunotherapy, or alternatively for stand-alone use in the development of therapies for the treatment, attenuation and/or prevention of allergy.

In particular, the inventors herein describe the utility of the TLR2 moieties described herein in treating allergy via the modulation of the phenotype of human allergen-specific Th2 cell-mediated immune responses and enhancement of counter-regulatory immune mechanisms. The inventors herein describe for the first time that:
The TLR2 moieties described herein are capable of suppressing allergen-driven Th2 cell responses through the suppression of cytokine production and T cell proliferation in human cells from allergic patients thus supporting the clinical relevance of the data;
Suppression of human allergen-driven Th2 cell responses is demonstrable when the TLR2 moiety is used as a stand-alone therapy or for use in conjunction with, or as an adjuvant with allergen immunotherapy (ie with an allergen);
The TLR2 moieties described herein are capable of suppressing a pathogenic Th2 cell-mediated immune response in different types of allergy including grass pollen allergy and cat allergy, thus demonstrating broad utility for the compounds in the treatment of different allergic diseases;
The effect of the TLR2 moieties described herein is not primarily dependent on upregulation of an allergen-specific Th1 cell-mediated immune response and does not result in substantial increases in the production of interferon gamma, thus differentiating the effect of the TLR2 moieties described herein from that of other TLR agonists, such as TLR4, TLR7/8 or TLR9;
The TLR2 moieties described herein are capable of promoting immune tolerance and homeostasis via the upregulation of IL-10 producing monocytes, T regulatory and/or B regulatory cells, antigen presenting cells or other immune cells; and
The effect of the TLR2 moieties described herein on suppression of allergen-driven pathologic Th2 cell-mediated responses and the up-regulation of tolerogenic counter-balancing immune mechanisms is superior to that of other tested TLR2 moieties disclosed herein.

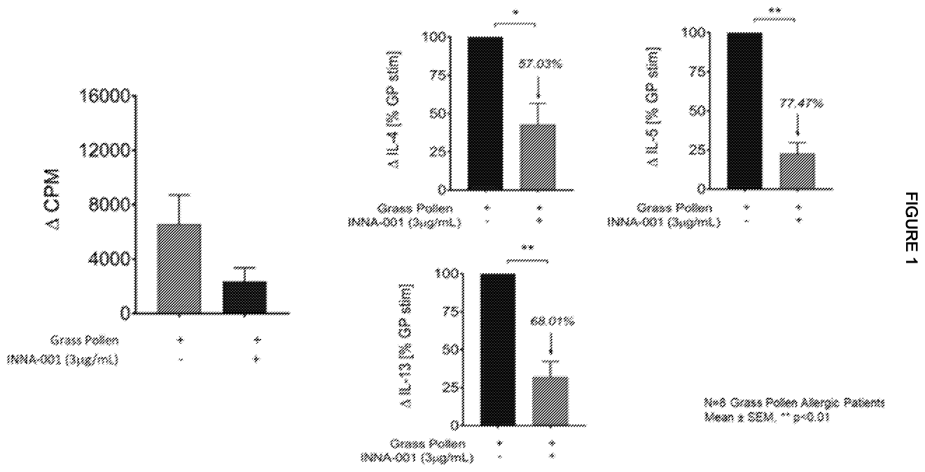

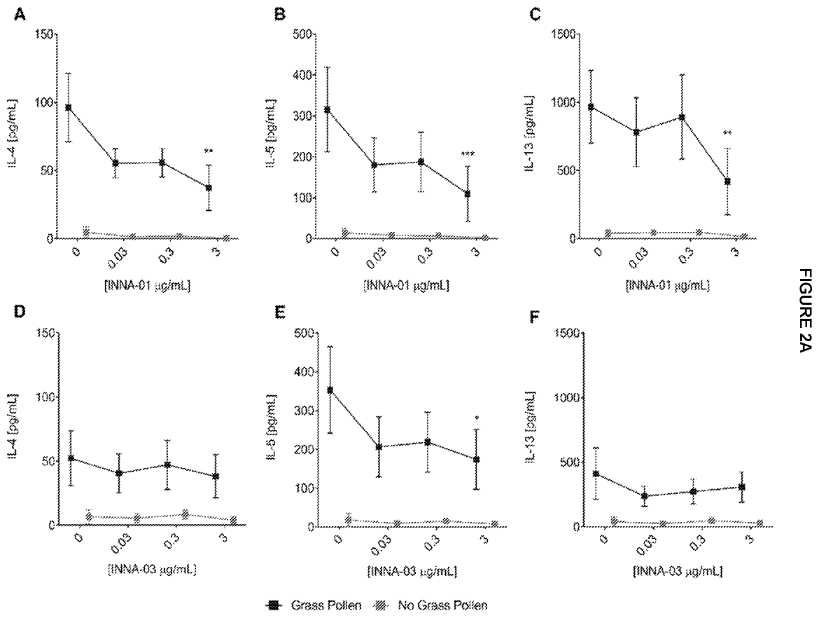

Importantly, the above observations were associated with a reduction of 50% or more of allergen-driven immune responses, as demonstrated by the reduced production of allergen-driven Th2 cytokines in peripheral blood mononuclear cells (PBMC) from allergic patients (ie as shown in FIG. 1). A person skilled in the art will appreciate that this is a strong indicator that the compounds described herein are likely to provide for an effective anti-allergy therapeutic approach.

Significantly, the compounds described herein have been compared to other TLR2 moieties including Pam2Cys-SS-PEG (alternatively denoted herein as INNA-003) and Pam2Cys-S-PEG (alternatively denoted herein as INNA-006). The TLR2 moieties described herein, when compared to the effect of Pam2Cys-SS-PEG or Pam2Cys-S-PEG, demonstrate:
greater reduction of allergen-stimulated Th2 cytokines;
greater increases of levels of IL-10;
no substantial effect on levels of interferon gamma (IFN gamma); and
greater expansion of the numbers of both naïve and memory T regulatory cells.

The TLR2 moieties described herein therefore provide for advantageous effects in the treatment of allergic conditions.

A skilled person will also understand that the invention extends to the use of any TLR2 moiety described herein in treating, attenuating and/or preventing an allergic response. As multiple types of allergy have been shown to be responsive to the TLR2 moieties described herein, a skilled person will also understand the applicability of the TLR2 moieties to various types of allergy driven by differing causative allergens and pathogeneses.

The findings described herein are significant as they establish new treatment regimes that have utility as a stand-alone therapy or for use in conjunction with, or as an adjuvant with allergen immunotherapy in humans. Moreover, such treatment regimes may promote desensitisation and immune tolerance in types of allergy that can be challenging to effectively treat. This work therefore identifies new treatment approaches that can act to prevent and treat allergy in patients suffering from different types of allergy by promoting desensitisation and/or immune tolerance and/or desensitisation in a patient against one or more types of allergens.

### Toll-Like Receptors (TLRs)

Toll Like Receptors (TLRs) are a family of glycoproteins that are widely expressed on immune and epithelial cells and act as “danger sensors” by permitting the quick recognition of molecular patterns conserved on the majority of microbes and the orchestration of host defence. Natural activation of TLRs triggers the upregulation of co-stimulatory molecules on antigen-presenting cells and generation of pro-inflammatory cytokines that mobilize both the innate and adaptive arms of immunity and instruct them to eliminate invading organisms, clear damaged self-tissues and preserve immune homeostasis. Stimulation of TLRs with synthetic agonists can reiterate in a controlled manner immunoregulatory effects analogous to those elicited by these conditions.

There are a number of TLRs that belong to this membrane receptor protein family including TLR1, TLR3, TLR4, TLR5, TLR6, TLR7, TLR8 and TLR9.

As used herein, the term “TLR2” is intended to mean Toll-Like Receptor 2 protein. In humans, TLR2 is encoded by the TLR2 gene. TLR2 is expressed on the surface of certain cells and plays a fundamental role in pathogen recognition and activation of innate immunity.

In accordance with the invention, there is provided use of a TLR2 moiety comprising, consisting essentially, or consisting of a TLR2 agonist and a polar polypeptide.

A TLR2 agonist is an agent that binds Toll-like receptor 2. The TLR2 agonist may bind to, and activate, TLR2 as a homodimer or heterodimer.

In an embodiment of the invention, the TLR2 agonist is lipid moiety. The lipid moiety may be selected from the group consisting of palmitolyl, myristroyl, steareoyl, lauroyl, decanoyl and combinations thereof.

In another embodiment, the TLR2 agonist is a lipopeptide in the form of Pam₂Cys, Pam₃Cys, Ste₂Cys, Lau₂Cys, and Oct₂Cys.

One of skill in the art would understand that the term “lipopeptide” means any composition of matter comprising one or more lipid moieties and one or more amino acid sequences that are conjugated. “Pam2Cys” (also known as dipalmitoyl-S-glyceryl-5 cysteine or S-[2, 3 bis(palmitoyloxy) propyl] cysteine corresponds to the lipid moiety of MALP-2, a macrophage-activating lipopeptide isolated from Mycoplasma fermentans.

Pam2Cys is known to be a ligand of TLR2 and has the structure:

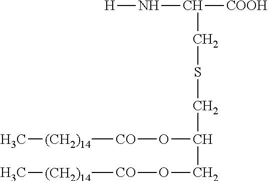

As used herein, reference to “S” as denoted in the above chemical structure defines a sulfur atom.

One of skill in the art will understand that the term “branched polypeptide” or “hyper-branched” polypeptide as used herein describes any branched or hyper-branched structure that may be suitably conjugated to Pam2Cys as understood by a skilled person in the art.

It will be understood that a “branched polypeptide” is a polymer that comprises a dendrite structure terminating in a plurality of up to four amino acids. It will be understood that a “hyper-branched polypeptide” is a polymer that comprises a dendrite structure terminating in a plurality of five, six, seven, eight or more amino acids.

A hyper-branched polypeptide may comprise a regularly ordered branch structure and/or an irregular branch structure. The hyper-branched polypeptide comprises a dendrite structure terminated in amino acids of the desired charge. For example, the dendrite structure is composed of lysine residues where further residues are attached to both the a and E amino groups. This is shown schematically below.

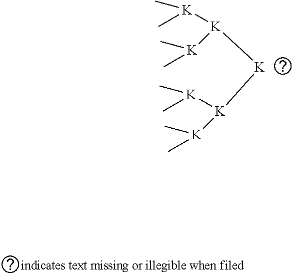

In an embodiment, the TLR2 agonist and the polar polypeptide are linked by at least one serine residue, preferably two serine residues.

In a preferred embodiment of the invention, the TLR2 moiety is Pam₂CysR₄ (alternatively denoted herein as INNA-001), having the structure:

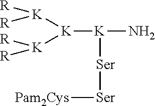

Pam₂CysR₄ comprises a hyper-branched peptide structure mediated by a scaffold of lysine (Lys or K) residues to which four arginine (Arg or R) residues are attached to give an overall net terminal charge of +8. The lipid moiety Pam₂Cys is conjugated on the ε-amino group of the C-terminal Lys residue through two serine (Ser or S) residues as described in WO 2016/037240 (PCT/AU2015/050540; US20170274068), which is herein incorporated by reference.

As used herein, the term “polar polypeptide” has its typical meaning as understood by a skilled person in the art and may include polypeptides with amino acid side chains with hydrogen donor and/or acceptor atoms. Such polar polypeptides may comprise the amino acids aspartic acid (Asp, D) glutamic acid (Glu, E), histidine (His, H), lysine (Lys, K), arginine (Arg, R), asparagine (Asn, N), glutamine (Gln, Q), serine (Ser, S), threonine (Thr, T) and tyrosine (Tyr, Y). In an embodiment, the polar polypeptide may act to increase the solubility of the TLR2 agonist in polar or aqueous solvents.

The polar polypeptide may comprise, consist essentially of, or consist of any branched or hyper-branched peptide described herein. Typically, the branched or hyper-branched peptide contains positively or negatively charged amino acids. Positively charged amino acids may be lysine, arginine, histidine, ornithine or combinations thereof. Negatively charged amino acids are glutamate or aspartate or combinations thereof. The branched or hyperbranched polypeptide may contain at least one lysine or arginine residue. Preferably, the charged amino acids are terminal, for example N-terminal. The branched peptides may have one of the following structures.

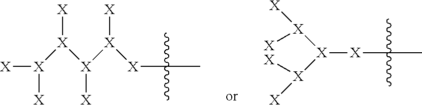

In the above structures X may independently be a charged residue, either a positively or negatively charged residue. Preferably, the positively charged amino acids are lysine, arginine, histidine or ornithine. Preferably, the negatively charged amino acids are glutamate or aspartate.

The polar polypeptide may be “R4”, a branched tetra arginine polypeptide; “R8”, a hyper-branched arginine polypeptide “H4”, a branched tetra histidine polypeptide; “H8”, a polypeptide containing hyper-branched histidine residues; “K4”, a polypeptide containing branched lysine residues; “K8”, a polypeptide containing hyper-branched lysine residues; “E4” a branched polypeptide containing glutamate residues and “E8” a hyper-branched polypeptide containing glutamate residues (see, e.g., “branched E8”, below). R4, H4, H8 and E8 have been previously described in WO/2010/115230 (PCT/AU2009/000469; US20150150966) and have the following structures:

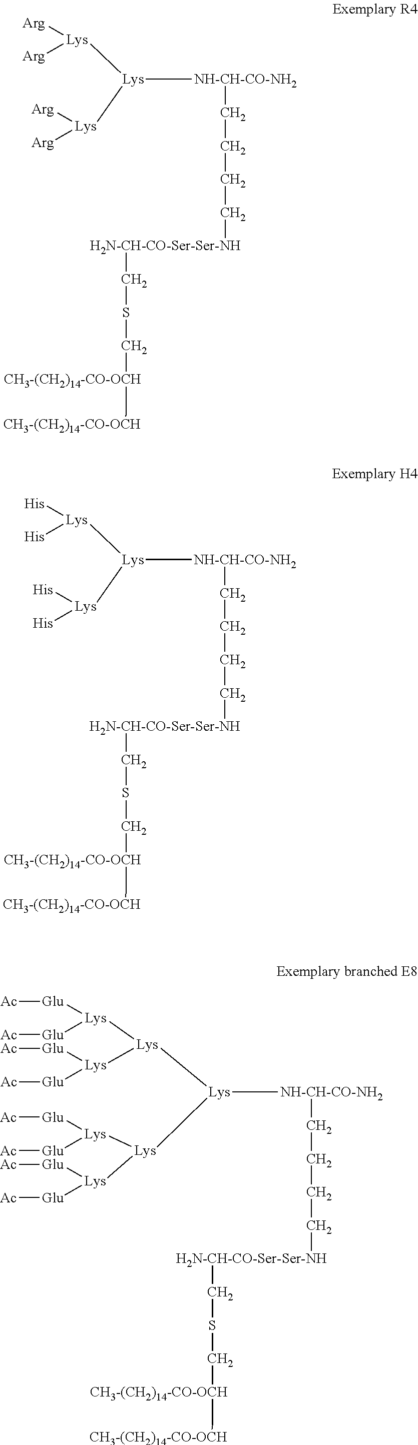

Following are schematic representations of some examples of branched (structures 1-5) immunogenic compositions comprising of positively charged (Arginine, R; Lysine, K) or negatively charged (Aspartic acid, D; Glutamic acid, E) amino acids in terminal positions such that their respective electrostatic charges are displayed to the environment. Each immunogenic composition also contains di palm itoyl-S-glyceryl cysteine (Pam2Cys) which is a ligand for Toll-Like Receptor 2. Two serine residues (Ser) are also incorporated. In the case of construct number 2, the peptide structure was assembled in the direction N→C, all other structures shown in the figure were assembled C→N. Positive and negative electrostatic charges are shown as 2−, 2+, 1−, 1+ etc. depending on the size of charge. Ac=acetyl group used to suppress the positive charge of alpha amino groups in the case of N-terminally situated Glutamic acid.

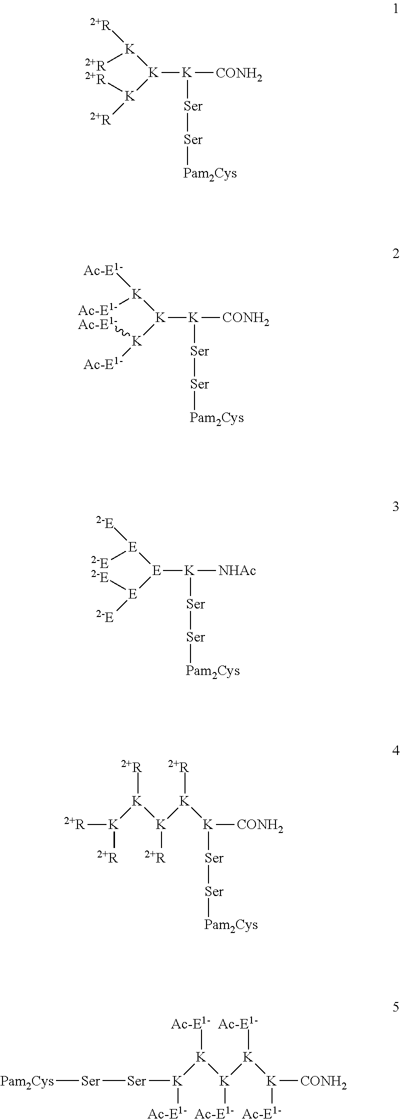

Other exemplary branched or hyper-branched polypeptides include:

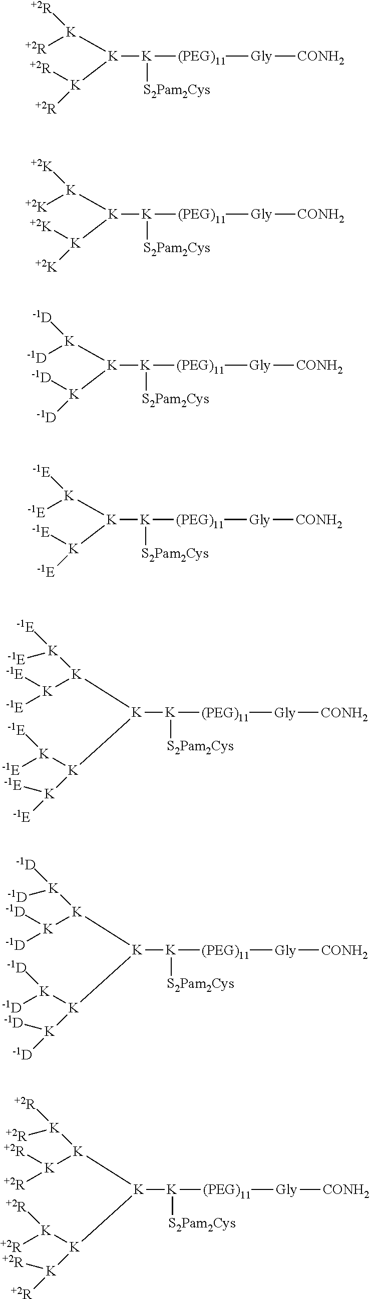

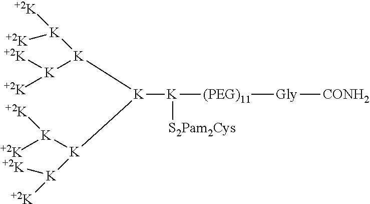

### Conjugation of a TLR2 Moiety to a Branched or Hyperbranched Peptide

The way in which the branched or hyper-branched polypeptide may be conjugated to a TLR2 moiety according to the present invention is well known to a person skilled in the art. For example, conjugation via F_{moc} chemistry, through a disulfide or a thioether bridge, or via oxime chemistry is envisaged and contemplated in the invention.

In an embodiment of the invention, the TLR2 moiety comprises Pam2Cys conjugated to a pendant R4 form. In a preferred form, pendant-Pam2Cys is conjugated to R4 according to the following structure:

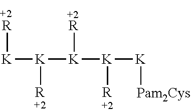

### Pharmaceutical Compositions

As used herein, reference to a TLR2 moiety and/or allergen also includes a pharmaceutically acceptable salt, solvate, polymorph or prodrug thereof.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae. All polymorphic forms of the compounds, salts, tautomers, N-oxides, solvates and/or prodrugs are within the scope of this invention and may be used in the methods of the invention.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably, the solvent used is a pharmaceutically acceptable solvent. Examples of suitable pharmaceutically acceptable solvents include, without limitation, water, ethanol and acetic acid. Most preferably, the solvent used is water. Solvates wherein the solvent is water may be referred to as hydrates of the compounds of the invention and salts or prodrugs thereof.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

Compounds described herein may demonstrate tautomerism. Tautomers are two interchangeable forms of a molecule that typically exist within an equilibrium. Any tautomers of a compound are to be understood as being within the scope of the invention and may be used in the methods of the invention.

The compounds described herein may contain one or more stereocentres. All steroisomers of the compounds are within the scope of the invention. Stereoisomers include enantiomers, diastereomers, geometric isomers (E and Z olephinic forms and cis and trans substitution patterns) and atropisomers. In some embodiments, the compound is stereoisomerically enriched at any stereocentre. The compound may be enriched in one stereoisomer over another by about 60, 70, 80, 90, 95, 98 or 99%.

The compounds as described herein are to also include isotope variations, such as the replacement of hydrogen for deuterium.

A “prodrug” is a compound that may not fully satisfy the structural requirements of the compounds provided herein, but is modified in vivo, following administration to a subject or patient, to produce a compound as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of any of the compounds described herein. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of the compounds described herein.

The general chemical terms used in the formulae herein have their usual meaning.

As used herein, ‘Ser’ refers to the amino acid serine and ‘Cys’ refers to the amino acid cysteine.

As used herein, the term “and/or” means “and”, or “or”, or both.

The term “(s)” following a noun contemplates the singular and plural form, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### Immune System

The immune system can be divided into: the (a) innate immune system comprised of components that provide for an immediate “first-line” of defence to counter pathogens and the (b) adaptive (acquired) immune system that includes the generation of antibodies and the production or stimulation of T-cells specifically designed to target particular pathogens. Via the utilisation of an adaptive immune response, the body can develop specific immunity to particular antigen(s) over time. This response takes days to develop, and so is not effective at preventing an initial invasion, but it will normally prevent any subsequent infection, and also aids in clearing up longer-lasting infections.

CD4+ lymphocytes, also called “helper” T cells, are immune response mediators, and play an important role in establishing and maximizing the capabilities of the adaptive immune response. Two types of effector CD4+T helper cell responses can be induced by antigen presenting cells (APC), designated Th1 and Th2, each designed to eliminate different types of pathogens. The factors that dictate whether an infection will trigger a Th1 or Th2 type response (type 1 or type 2 response) are not fully understood, but the response generated plays an important role in the clearance of different pathogens.

The Th1 cell-mediated response is characterized by the production of interferon-gamma, which activates the bactericidal activities of macrophages, and induces B cells to make opsonizing (coating) and complement-fixing antibodies, and leads to “cell-mediated immunity”.

The Th2 cell-mediated response is characterized by the release of interleukin 4, interleukin 5 and interleukin-13, which results in the activation of B cells to make neutralizing non-cytolytic antibodies, leading to “humoral immunity”. Generally, Th1 responses are more effective against intracellular pathogens (viruses and bacteria that are inside host cells), while Th2 cell-mediated responses are more effective against extracellular bacteria, parasites including helminths and toxins. Like cytotoxic T cells, most of the CD4+ helper cells will die upon resolution of infection, with a few remaining as CD4+ memory cells. Th2 cells contribute to chronic inflammatory disorders, including allergy and asthma.

Monocytes are innate blood cells involved in the maintenance of immune homeostasis. They are early responders to pathogens in acute infections and are involved in tissue injury. There are different types of monocytes. The most predominant are the “classical monocytes”, which express high levels of CD14 on their surface, are devoid of surface CD16 and account for approximately 80% of the total monocyte population. The remaining 20% are “non-classical monocytes,” characterized by very low expression of surface CD14 and high levels of CD16. Classical monocytes are phagocytic, can differentiate into macrophages in tissue and can contribute to chronic disease. Non-classical/patrolling monocytes have been widely viewed as anti-inflammatory, as they maintain vascular homeostasis. They are a first line of defence in recognition and clearance of pathogens and they have antigen-presenting properties. Their roles in chronic disease are less clear. Induction of IL-10 by monocytes is associated with inhibition of antigen presentation and stimulation of endocytic activity. IL-10 inhibits dendritic cell differentiation from monocytes and, in a substantial proportion of the cells, promotes the differentiation to mature macrophages.

### Allergens

As used herein, the term “allergen” will be understood to mean any substance that is recognized by the immune system and causes an allergic reaction.

Allergens are common innocuous environmental proteins that are capable of inducing in predisposed individuals (allergic subjects) the generation of allergen-specific Th2 cells with an effector phenotype. The Th2 cells, through the cytokines they secrete (IL-4, IL-5, IL-13 etc) drive two critical elements responsible for the manifestation of allergic symptoms: a) the increased synthesis of allergen-specific IgE, which mediates the type I hypersensitivity reactions, through acute activation of mast cells and basophils and release of histamine, prostaglandins and leukotrienes and b) the differentiation, survival and chemotaxis of pro-inflammatory cells, namely mast cells, basophils and eosinophils, that orchestrate the development of chronic inflammation. In certain instances, the acute and/or chronic inflammatory allergic response is local, in the organ of allergen entry (i.e. in the nose (rhinitis). In other instances, the acute inflammatory allergic response is systemic and results in the narrowing of airways (i.e., bronchoconstriction), vasodilation, development of skin rash or oedema etc (i.e., anaphylaxis). Thus, it is contemplated that the compounds and compositions disclosed herein will have utility in suppressing one or more symptoms of inflammation associated with an allergic immune response.

In an embodiment of the invention, the compounds and/or compositions described herein are useful for the treatment of allergen-driven hypersensitivity.

At a broad level, a skilled person will understand that conditions to be treated with the compounds and compositions described herein include microbe allergies (e.g., bacteria, virus, fungi), pollen allergies (e.g., farm plant, tree, weed, grass), food allergies, animal dander allergies, hymenoptera venom allergies, mite allergies (e.g. house dust mites), skin allergies (ie latex), drug allergies, plant allergies, mould allergy, allergic asthma and animal allergies.

Non-limiting examples of allergens that are associated with the above-described allergies or conditions to be treated that fall within the scope of the current invention include the following. It will also be understood that one or more of the following allergens may be used in combination with a TLR2 agonist comprising a branched peptide described herein.

In an embodiment of the invention, the allergen can be selected from one or more types of mites, e.g., Mite, House Dust (Dermatophagoides farinae); Mite, House Dust (Dermatophagoides pteronyssinus); Mite, Food/Storage (Acarus siro); Mite, House Dust (Blomia tropicalis); Mite, Storage (Chortoglyphus arcuates); Mite, House Dust (Euroglyphus maynei); Mite, Food/Storage (Lepidoglyphus destructor); Mite, Food/Storage (Tyrophagus putrescentiae); and Mite, House Dust (Glycyphagus domesticus).

In an embodiment of the invention, the allergen can be selected from one or more types of venoms, e.g., Bumble Bee Venom (Bombus spp.); European Hornet Venom (Vespa crabro); Honey Bee (Apis mellifera.); Mixed Hornet Venom (Dolichovespula spp); Mixed Paper Wasp Venom (Polistes spp.); Mixed Yellow Jacket Venom (Vespula spp.); White (bald)-faced Hornet Venom (Dolichovespula maculate); and Yell ow Hornet Venom (Dolichovespula arenaria).

In an embodiment of the invention, the allergen can be selected from one or more types of insects, e.g., Ant, Carpenter (Camponotus pennsylvanicus); Ant, Fire (Solenopsis invicta); Ant, Fire (Solenopsis richteri); Cockroach, American (Periplaneta Americana); Cockroach, German (Blattella germanica); Cockroach, Oriental (Blatta orientalis); Horse Fly (Tabanus spp.); House Fly (Musca domestica); Mayfly (Ephemeroptera spp.); Mosquito (Culicidae sp.); and Moth (Heterocera spp.).

In an embodiment of the invention, the allergen can be selected from one or more types of epithelia, dander, and hair and feathers, e.g., Canary Feathers (Serinus canaria); Cat Epithelia (Felis domesticus)); Cattle Epithelia (Bos Taurus); Chicken Feathers (Gallus gallas (domesticus)); Dog Epithelia, Mixed Breeds (Canis familiaris); Duck Feathers (Anal platyrhynchos); Gerbil Epithelia (Meriones unguiculatus); Goat Epithelia (Capra hircus); Goose Feathers (Anser domesticus); Guinea Pig (Cavia porcellus); Epithelia ((cobaya)); Hamster Epithelia (Mesocricetus auratus); Hog Epithelia (Sus scrofa); Horse Epithelia (Equus caballus); Mouse Epithelia (Mus musculus); Parakeet Feathers (Psittacidae spp.); Pigeon Feathers (Columbafasciata); Rabbit Epithelia (Oryctolagus cuniculus); Rat Spithelia (Rettus norvegicus); and Wool, Sheep (Ovis aries).

In an embodiment of the invention, the allergen can be selected from one or more types of dander, e.g., Cat dander/Antigen (Felis catus (domesticus)); Dog Dander, Mixed-Breed (Canis familiaris); and Poodle Dander (Canisfamiliaris).

In an embodiment of the invention, the allergen can be selected from one or more types of fungi, e.g., Acremonium strictum; Alternaria alternate; Aspergillus amstelodami; Aspergillus flavus; Aspergillus furmigatus; Aspergillus nidulans; Aspergillus niger; Aspergillus terreus; Aspergillus versicolor; Aureohasidium Pullulans; Bipolaris sorokiniana; Botrytis cinerea; Candida albicans; Chaetomium globosum; Cladosporium herbarum; Cladosporium sphaerospermum; Drechslere spicifera; Epicoccum mgrum; Epidermophyton floccosum; Fusarium moniliforme; Fusarium solani; Geotrichum candidum; Gliocladium viride; Helminthosporium solani; Microsporum canis; Cephalosporium acremonium; Alternaria fermis; Aspergillus glaucus; Pullularia pullulans; Drechslera sorokiniana; Helminthosporium sativum; Hormodendrum hordei; Curvularia spicifera; Epicoccum purpurascens; Oospora lactic; Gliocladium deliquescens; Spondylocladium atrovirens; Microsporum lanosum; Mucor circinelloides f. circinelloides; Mucor circinelloides f. lusitanicus; Muncor plumbeus; Mycogone perniciosa; Neurospora intermedia; Nigrospora oryzae; Paecilomyces variotii; Penicillium brevi-compactum; Penicillium camembertii; Penicillium chrysogenum; Penicillium digitatum; Penicillium expensum; Penicillium notatum; Penicillium roquefortii; Phoma betae; Phomma herbarum; Rhigopus oryzae; Rhizopus stolonifer; Rhodotorula mucilaginosa; Saccharomyces cerevisiae; Scopulariopsis brevicaulis; Serpula lacrymans; Setosphaeria rostrata; Stemphylium hotryosum; Stemphylium solani; Trichoderma harzianum; Trichophyton mentagrophytes; Trichophyton rubrum; Trichothecium roseum; Mucor mucedo; Mucor racemosus; Neurospora sitophil; Monilia sitophila; Phoma pigmentivora; Rhizopus arrhizus; Rhizopus nigricans; Rhodotorula rubra var. mucilaginosa; Merulius lacrymans; Exserohilum rostratum; Helminthosporium halodes; Trichoderma viride; Trichophyton interdigitale; and Cephalothecium roseum.

In an embodiment of the invention, the allergen can be selected from one or more types of smuts, e.g., Barley Smut (Ustilago nuda); Bermuda Grass (ustilago); Smut (cynodontis); Corn Smut (Ustilago maydis); Johnson Grass (Sporisorium); Smut (cruentum); Oat Smut (Ustilago avenae); and Wheat Smut (Ustilago tritici).

In an embodiment of the invention, the allergen can be selected from one or more types of grass pollens, e.g., Bahia (Paspalum notalum); Bermuda (Cynodon dactylon) Blue, Canada (Paa compressa); Brame, Smooth (Bromus inermis); Canary (Phalaris arundinacea); Corn (Zea mays); Couch/Quack (Elytrigia repens (Agropyron repens)); Johnson (Sorghum halepense); Kentucky Blue (Paa pratensis); Meadow Fescue (Festuca pratensis (elatior)); Oat, Cultivated (Avena sativa); Orchard (Dactylis glomerata); Red Top (Agrostis gigantean (alba)); Rye, Cultivated (Secale cereale); Rye, Giant Wild (Leymus (Elymus) condensatus); Rye, Italian (Lolium perenne ssp. Multiflorum); Rye, Perennial (Lolium perenne); Sweet Vernal (Anthoxanehum odoratum); Timothy (Phleum pretense); Velvet (Holcus lanatus); Wheat, Cultivated (Triticum aestivum); St. Augustine grass (Stenotaphrum secundatum), and Wheatgrass, Western (Elymus (Agropyron).

In an embodiment of the invention, the allergen can be selected from one or more types of weed pollens, e.g., Allscale (Atriplex polycarpa); Baccharis (Baccharis halimifolia); Baccharis (Baccharis sarothroides); Burrobrush (Hymenoclea salsola); Careless Weed (Amaranthus hybridus); Cocklebur (Xanthium strumarium (commune)); Dock, Yellow (Rumex crispus); Dog Fennel (Eupatorium capillifolium); Goldenrod (Solidago spp.); Hemp, Western Water (Amaranthus tuberculatus (Acnida tamariscina)); Iodine Bush (Allenrolfea occidentalis); Jerusalem Oak (Chenopodium botrys); Kochia/Firebush (Kochia scoparia); Lambs Quarter (Chenopodium album); Marsh Elder, Burweed (Iva xanthifolia); Marsh Elder, Narrowleaf (Iva angustifolia); Marsh Elder, Rough (Iva annua (ciliata)); Mexican Tea (Chenopodium ambrosioides); Mugwort, Common (Artemisia vulgaris); Mugwort, Darkleaved (Artemisia ludoviciana); Nettle (Urtica dioica); Palmer's Amaranth (Amaranthus palmeri); Pigweed, Redroot/Rough (Amaranthus retroflexus); Pigweed, Spiny (Amaranthus spinosus); Plantain, English (Plantago lanceolata); Poverty Weed (Iva axillaris); Quailbrush (Atriplex lentiformis); Rabbit Bush (Ambrosia deltoidea); Ragweed, Desert (Ambrosia dumosa); Ragweed, False (Ambrosia acanthicarpa); Ragweed, Giant (Ambrosia trifida); Ragweed, Short (Ambrosia artemisiifolia); Ragweed, Slender (Ambrosia confertiflora); Ragweed, Southern (Ambrosia bidentata); Ragweed, Western (Ambrosia psilostachya); Russian Thistle (Salsola kali (pestifer)); Sage, Coastal (Artemisia californica); Sage, Pasture (Artemisia frigida); Sagebrush, Common (Artemisia tridentate); Saltbush, Annual (Atriplex wrightii); Shadscale (Atriplex confertifolia); Sorrel, Red/Sheep (Rumex acetosella); and Wingscale (Atriplex canescens); Wormwood, Annual (Artemisia annua).

In an embodiment of the invention, the allergen can be selected from one or more types of tree pollens, e.g., Acacia (Acacia spp.); Alder, European (Alnus glutinosa); Alder, Red (Alnus rubra); Alder, Tag (Alnus incana ssp. Rugosa): Alder, White (Alnus rhombifolia); Ash, Arizona (Fraxinus velutina); Ash, Green/Red (Fraxinus pennsylvanica); Ash, Oregon (Fraxinus latifolia); Ash, White (Fraxinus Americana); Aspen (Populus tremuloides); Bayberry (Myrica cerifera); Beech, American (Fagus grandifolia (americana)) Beefwood/Australian Pine (Casuarina equisetifolia); Birch, Black/Sweet (Betula lenta); Birch, European White (Betula pendula); Birch, Red/River (Betula nigra); Birch, Spring (Betula occidentalis (jontinalis)); Birch, White (Betula populifolia); Box Elder (Acer negundo); Cedar, Japanese (Cryptomeria japonica); Cedar, Mountain (Juniperus ashei (sabinoides)); Cedar, Red (Juniperus virginiana); Cedar, Salt (Tamarix gallica); Cottonwood, Black (Populus balsamifera ssp. Trichocarpa); Cottonwood, Eastern (Populus deltoids); Cottonwood, Fremont (Populus fremontii); Cottonwood, Rio Grande (Populus wislizeni); Cottonwood, Western (Populus monilifera (sargentii)); Cypress, Arizona (Cupressus arizonica); Cypress, Bald (Taxodium distichum); Cypress, Italian (Cupressus sempervirens); Elm, American (Ulmus Americana); Elm, Cedar (Ulmus crassifolia); Elm, Siberian (Ulmus pumila); Eucalyptus (Eucalyptus globulus); Hackberry (Celtis occidentalis); Hazelnut (Corylus Americana); Hazelnut, European (Corylus avellana); Hickory, Pignut (Carya glabra); Hickory, Shagbark (Carya ovata); Hickory, Shellbark (Carya laciniosa); Hickory, White (Carya alba); Juniper, Oneseed (Juniperus monosperma); Juniper, Pinchot (Juniperus pinchotii); Juniper, Rocky Mountain (Juniperus scopulorum); Juniper, Utah (Juniperus osteosperma); Juniper, Western (Juniperus occidentalis); Locust Blossom, (Robinia); Black (pseudoacacia); Mango Blossom (Mangifera indica); Maple, Coast (Acer macrophyllum); Maple, Red (Acer rubrum); Maple, Silver (Acer saccharinum); Maple, Sugar (Acer saccharum); Melaleuca (Melaleuca quinquenervia (leucadendron)); Mesquite (Prosopis glandulosa (julifiora)); Mulberry, Paper (Broussonetia papyrifera); Mulberry, Red (Marus rubra); Mulberry, White (Marus alba); Oak, Arizona/Gambel (Quercus gambeiji); Oak, Black (Quercus velutina); Oak, Bur (Quercus macrocarpa); Oak, California Black (Quercus kelloggii); Oak, California Live (Quercus agrifolia); Oak, California White/Valley (Quercus lobata); Oak, English (Quercus robur); Oak, Holly (Quercus ilex); Oak, Post (Quercus stellata); Oak, Red (Quercus rubra); Oak, Scrub (Quercus dumosa); Oak, Virginia Live (Quercus virginiana); Oak, Water (Quercus nigra); Oak, Western White/Gany (Quercus garryana); Oak, White (Quercus alba); Olive (Olea europaea); Olive, Russian (Elaeagnus angustifolia); Orange Pollen (Citrus sinensis); Palm, Queen (Arecastrum romanzoffianum (Cocos plumosa)); Pecan (Carya illinoensis); Pepper Tree (Schinus molle); Pepper Tree/Florida Holly (Schinus terebinthifolius); Pine, Loblolly (Pinus taeda); Pine, Eastern White (Pinus strobus); Pine, Longleaf (Pinus palustris); Pine, Ponderosa (Pinus ponderosa); Pine, Slash (Pinus elliottii); Pine, Virginia (Pinus virginiana); Pine, Western White (Pinus monticola); Pine, Yell ow (Pinus echinata); Poplar, Lombardy (Populus nigra); Poplar, White (Populus alba) Privet (Ligustrum vulgare); Sweet Gum (Liquidambar styraciflua); Sycamore, Eastern (Platanus occidentalis); Sycamore, Oriental (Platanus orientalis); Sycamore, Western (Platanus racemosa); Sycamore/London Plane (Platanus acerifolia); Walnut, Black (Juglans nigra); Walnut, California Black (Juglans californica); Walnut, English (Juglans regia); Willow, Arroyo (Salix lasiolepis); Willow, Black (Salix nigra); and Willow, Pussy (Salix discolor).

In an embodiment of the invention, the allergen can be selected from one or more types of wild and cultivated flowers, e.g., Daisy, Ox-Eye (Chrysanthemum leucanthemum); Dandelion (Taraxacum officinale); and Sunflower (Helianthus annuus).

In an embodiment of the invention, the allergen can be selected from one or more types of cultivated farm plant pollens, e.g., Alfalfa (Medicago sativa); Castor Bean (Ricinus communis); Clover, Red (Trifolium pratense); Mustard (Brassica spp.); and Sugar Beet (Beta vulgaris).

In an embodiment of the invention, the allergen can be selected from one or more types of plant food, e.g., Almond (Prunus dulcis); Apple (Malus pumila); Apricot (Prunus armeniaca); Banana (Musa paradisiaca (sapientum)); Barley (Hordeum vulgare); Bean, Lima (Phaseolus lunates); Bean, Navy (Phaseolus vulgaris); Bean, Pinto (Phaseolus sp.) Bean, Red Kidney (Phaseolus sp.); Bean, String/Green (Phaseolus vulgaris); Blackberry (Rubus allegheniensis); Blueberry (Vaccinium sp.); Broccoli (Brassica oleracea var. botrytis); Buckwheat (Fagopyrum esculentum); Cabbage (Brassica oleracea var. capitata); Cacao Bean (Theobroma cacao); Cantaloupe (Cucumis melo); Carrot (Daucus carota); Cauliflower (Brassica oleracea var. botrytis); Celery (Apium graveolens var. dulce); Cherry (Prunus sp.); Cinnamon (Cinnamomum verum); Coffee (Coffee Arabica); Corn (Zea mays); Cranberry (Vaccinium macrocarpon); Cucumber (Cucumis sativus); Garlic (Allium sativum); Ginger (Zingiber officinale); Grape (Vitis sp.); Grapefruit (Citrus paradise); Hops (Humulus lupulus); Lemon (Citrus Ziman); Lettuce Malt (Lactuca sativa); Mushroom (Agaricus campestris); Mustard (Brassica sp.); Nutmeg (Myristic fragrans); Oat (Avena sativa); Olive, Green (Olea europaea); Onion (Allium cepa var. cepa); Orange (Citrus sinensis); Pea, Blackeye (Vigna unguiculata); Pea, Green (Pisum sativum (English)); Peach (Prunus persica); Pear (Pyrus communis); Pepper, Black (Piper nigrum); Pepper, Green (Capsicum annuum var. annuum); Pineapple (Ananas comosus); Potato, Sweet (Ipomoea batatas); Potato, White (Solanum tuberosum); Raspberry (Rubus idaeus var. idaeus); Rice (Oryza sativa); Rye (Secale cereale); Sesame Seed (Sesamum orientale (indicum)); Soybean (Glycine max); Spinach (Spinacia oleracea); Squash, Yellow (Cucurbita pepo var. melopepo); Strawberry (Fraearia chiloensis); Tomato (Lycopersicon esculentum (lycopersicum)); Turnip (Brassica rapa var. rapa); Vanilla Bean (Vanilla planifolia); Watermelon (Citrullus lanatus var. lanatus); and Wheat, Whole (Triticum aestivum).

In an embodiment of the invention, the allergen can be selected from one or more types of fish and shellfish, e.g., Bass, Black (Micropterus sp.); Catfish (Ictalurus punctatus); Clam (Mercenaria mercenaria); Codfish (Gadus morhua); Crab (Callinectes sapidus); Flounder (Platichthys sp.); Halibut (Hippoglossus sp.); Lobster (Homarus americanus); Mackerel (Scomber scombrus); Oyster (Crassostrea virginica); Perch (Sebastes marinus); Salmon (Salmo salar); Sardine (Clupeiformes); Scallop (Pectan magellanicus); Shrimp (Penaeus sp.); Trout, Lake (Salvelinus sp.); and Tuna Fish (Thunnus sp.).

In an embodiment of the invention, the allergen can be selected from one or more types of animal foods, e.g., Beef (Bus Taurus); Lamb (Ovis aries); and Pork (Sus scrofa).

In an embodiment of the invention, the allergen can be selected from one or more types of poultry products, e.g., Chicken (Gallus gallus); Egg, Chicken, White (Gallus gallus); Egg, Chicken, Yolk (Gallus gallus); and Turkey (Meleagris gallopavo).

In an embodiment of the invention, the allergen can be selected from one or more types of dairy products, e.g., Casein, bovine (Bos Taurus) and Milk, bovine (Bos Taurus).

In an embodiment of the invention, the allergen can be selected from one or more types of nuts, e.g., Brazil Nut (Bertholletia excelsa); Cashew Nut (Anacardium occidental); Coconut (Cocos nucifera); Filbert/Hazelnut (Corylus Americana); Peanut (Arachis hypogaea); Pecan (Carya illinoensis); Walnut, Black (Juglans nigra); and Walnut, English (Juglans regia).

In an embodiment of the invention, the allergen can be selected from one or more types of miscellaneous materials, e.g., latex, silver, or the like.

In an embodiment of the invention, the allergen may be an allergen derivative including but not limited to hypo-allergens, recombinant allergens, peptides from allergens.

In a preferred embodiment, the allergen is selected from the group consisting of Timothy grass pollen (Phleum pratense) or the cat allergen Fel d 1 which is typically produced in cat saliva and sebaceous glands.

Typically, a broad range of symptoms will be associated with the above-mentioned allergens including sneezing, itchy, runny or blocked nose, itchy, red, watering eyes, wheezing and/or chest tightness, reduced shortness of breath and/or coughing, swelling (eg of lips), nausea, vomiting and/or diarrhoea, rash development and itchiness. In severe cases, anaphylaxis may develop where an individual may present with one or more of swelling of throat, difficulty in breathing, light-headedness, cyanosis of lips and/or skin and collapsing and/or losing consciousness. In the most severe cases, the allergen may be fatal and cause death of the individual.

The existence of, improvement in, or treatment of allergy may be determined by any clinically or biochemically relevant method as described herein or known in the art including allergy skin tests, nasal, inhaled, conjunctival or food allergen provocation or challenge, changes in the levels of allergen-specific immunoglobulins, including IgE, or other allergy-related biomarkers. A positive response to treatment of an allergy may be determined by any method known in the art and may include the determination of:
reduced sneezing;
reduced itchy, runny or blocked nose;
reduced itchy, red, watering eyes;
reduced wheezing and/or chest tightness, reduced shortness of breath and/or coughing;
reduced swelling (eg of lips);
reduced nausea, vomiting and/or diarrhoea;
reduced rash development and itchiness;
reduced anaphylaxis including reduced swelling of throat, reduced difficulty in breathing, reduced light headedness, reduced cyanosis of lips and/or skin and prevention of collapsing and/or losing consciousness and/or death.

The determination of any of the above may be considered to be a positive response to the compounds and/or compositions described herein. Alternatively, a reduction in the requirement for standard allergy medication that offers symptomatic relief may be considered to be a positive response to the compounds and/or compositions described herein

In embodiments where the compounds and/or compositions described herein prevent an allergic response, a skilled person will understand that the above-mentioned symptoms will be minimised or prevented.

In embodiments where the compounds and/or compositions described herein are capable of desensitising and/or tolerising a subject to an allergen, a skilled person will understand that the above-mentioned symptoms will be prevented.

The subject who has received treatment for allergy may be partially or completely treated, or they may be desensitised and/or able to tolerate exposure to higher amounts of allergen. A skilled person will understand desensitisation or tolerance to an allergen(s) to relate to the prevention of an aberrant immune response against the particular antigen. Allergen-desensitisation or tolerance may be achieved via the enhanced presence or function of regulatory B and/or T cells. In the context of the current invention, desensitisation or tolerance to allergens may be achieved by administering to the subject a TLR2 moiety described herein with or without an allergen(s). When administered with an allergen, the TLR2 agonist is capable of helping to stimulate and enhance the adaptive immune response against the allergen.

It will be understood that desensitisation to an allergen refers to the temporary state of non-clinical reactivity upon exposure to the allergen. Tolerance to an allergen is used to describe the persistent or permanent clinical non-reactivity upon exposure to the allergen that is mediated by long-lasting suppression of aberrant adaptive immune responsiveness to the specific allergen.

In an embodiment of the invention, the subject, having received a treatment for a given allergy, as described above, may have a 10%, 20%, 30%, 40%, 50%, 60%, 70%, 80%, 90% or greater reduction in the measurable parameters of any of those symptoms described above, as may be determined upon physical examination or by clinical tests including allergy skin tests, nasal, inhaled, conjunctival or food allergen provocation or challenge, changes in the levels of allergen-specific Immunoglobulins, including IgE, or other allergy-related biomarkers. Alternatively, where the subject is completely desensitised or tolerised, there may be a complete, lasting disappearance of all detectable manifestations of the allergy, such that the subject does not have any detectable signs of allergy according to those parameters described above or others known in the art. The subject may have substantially undetectable signs of allergy. An allergy that is “substantially undetectable” generally refers to a circumstance where therapy has depleted the extent, severity or other physical measure of an allergy so that through using relevant standard assessment techniques known in the art to determine the presence of one or more symptoms described above, the allergy, as a consequence of the treatment, is not clearly detectable.

The outcome of treatment with the compounds and/or compositions described herein may be to reduce allergen-driven Th2 cell-mediated responses; promote immune tolerance and homeostasis via the regulation of IL-10 producing monocytes, induction of T regulatory and B regulatory cells and relieve to some extent one or more of the symptoms associated with the allergy described above, and/or promote survival.

In one embodiment, the method is particularly useful for preventing anaphylaxis-induced collapse and death of a subject. Thus, in one embodiment, the method is particularly useful for extending survival of the subject, including overall survival as well as progression free survival. It will be understood that overall survival is the length of time from either the date of diagnosis or the start of treatment of an allergy, that patients diagnosed with the allergy are still alive. It will be understood that progression free survival is the length of time during and after the treatment of an allergy that a patient lives with the allergy but it does not get worse.

Survival analysis can be performed using well-known techniques in the art including the Kaplan-Meier method. The Kaplan-Meier method estimates the survival function from life-time data. In medical research, it can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In one embodiment, the method is particularly useful for providing a complete response to treatment whereby all signs of allergy in response to treatment have disappeared.

### Administration and Dosage

In an embodiment of the invention, therapeutically effective amounts of a TLR2 moiety described herein are administered to the subject. In alternate embodiments, therapeutically effective amounts of a (i) TLR2 moiety described herein and an (ii) allergen(s) described herein are administered to the subject.

Administering refers to the physical introduction of a composition comprising a therapeutic agent to a subject, using any of the various methods and delivery systems known to those skilled in the art including those described herein. Pharmaceutical compositions may be formulated from compounds of the invention as described herein for any appropriate route of administration. Typically, in addition to the therapeutic agent (eg a TLR2 moiety and/or allergen), a pharmaceutical composition comprises a pharmaceutically acceptable excipient, carrier and/or diluent. Examples of suitable components for inclusion in a pharmaceutical composition are described in Martindale—The Extra Pharmacopoeia (Pharmaceutical Press, London 1993) and Martin (ed.), Remington's Pharmaceutical Sciences.

Suitable routes of administration for implementing the defined methods include oral, sublingual, intravenous, intramuscular, subcutaneous, intraperitoneal, spinal or other parenteral routes of administration, for example by injection or infusion, as well as topical, epidermal and intradermal administration. Treatment via the respiratory system (inhalation or intranasally) is also contemplated. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion, as well as in vivo electroporation. Administering can also be performed, for example, once, a plurality of times, and/or over one or more extended periods.

The phrase ‘therapeutically effective amount’ or ‘effective amount’ generally refers to an amount of a TLR2 moiety and/or allergen, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

For instance, for the treatment, attenuation or prevention of allergy, a therapeutically effective amount of the compounds or compositions described herein can inhibit an allergic response by at least about 10%, by at least about 20%, by at least about 30%, by at least about 40%, by at least about 50%, by at least about 60%, by at least about 70%, by at least about 80%, or by at least about 90% or more, relative to untreated subjects.

Alternatively, the treatments described herein may cause desensitisation or tolerisation of the subject to a given allergen, as described herein. A therapeutically effective amount of a drug may also include a “preventative” or “prophylactically effective amount,” which is any amount of the TLR2 moiety and/or allergen administered to a subject at risk of developing an allergy (including an anaphylactic response) or with a history of developing one or more symptoms to a given allergen. In certain embodiments, the prophylactically effective amount prevents the development or recurrence of the allergy entirely and therefore effectively desensitises and tolerises the subject. “Inhibiting” or “preventing” the development or recurrence of an allergic response means either lessening the likelihood of the allergy's development or recurrence, or preventing the development or recurrence of the allergy entirely.

The exact amount of the therapeutically effective amount of the TLR2 moiety and/or allergen required may vary from subject to subject, depending on the species, age and general condition of the subject, mode of administration and the like. Thus, it may not be possible to specify an exact therapeutically effective amount. However, an appropriate therapeutically effective amount in any individual case may be determined by one of ordinary skill in the art using routine experimentation. In one aspect, the dose administered to a subject is any therapeutically effective amount that reduces symptoms associated with the allergy as a result of any one of sneezing, itchy, runny or blocked nose; itchy, red, watering eyes; wheezing and/or chest tightness; shortness of breath and/or coughing, swelling (eg of lips), nausea, vomiting and/or diarrhoea; rash development and itchiness; swelling of throat; difficulty in breathing; light headedness; cyanosis of lips and/or skin and collapsing and/or losing consciousness, as described herein. Additionally or alternatively, the therapeutically effective amount may lead to increased survival of the subject.

In some embodiments, a therapeutically effective amount of a TLR2 moiety for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound. Dosage regimes are adjusted to suit the exigencies of the situation and may be adjusted to produce the optimum therapeutic dose.

In any aspect of the invention, the amount of any allergen described herein may be in the range of from about 0.1 to about 20 ng, about 0.1 to about 10 ng, about 0.1 to about 5 ng, about 1 to about 5 ng, about 2 to about 5 ng, about 7.5 to about 12.5 ng, about 0.1 to about 30 ng, about 0.1 to about 100 ng, about 0.1 to about 500 ng, about 0.1 to about 1000 ng, about 0.1 to about 2000 ng, about 0.1 to about 100 μg, about 0.1 to about 250 μg, about 0.1 to about 500 μg, about 0.1 to about 750 μg, about 0.1 to about 1000 μg, about 0.1 to about 0.25 mg, about 0.1 to about 0.5 mg, about 0.1 to about 0.75 mg, about 0.1 to about 1.0 mg or about 0.1 to about 1.25 mg or about 0.1 to about 1.5 mg. For example, dosages can be about 0.1, about 0.3, about 1, about 2, about 3, about 5 or about 10 ng, or, about 0.3, about 1, about 2, about 3, or about 5 ng. In another aspect of the invention, the amount of any allergen described herein may be in the range of about 0.5 to 5 ng, preferably in the range of about 1 to 3 ng, more preferably 1.23-2.46 ng. In another embodiment, the amount of any allergen described herein may be in the range of about 0.1 ng to 1 mg.

Dosage regimes are adjusted to suit the exigencies of the situation and may be adjusted to produce the optimum therapeutic dose.

Typically, a therapeutically effective dosage is formulated to contain a concentration (by weight) of at least about 0.1% up to about 50% or more, and all combinations and sub-combinations of ranges therein. The compositions can be formulated to contain one or more compounds, or a pharmaceutically acceptable salt, polymorph or prodrug thereof in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

In some embodiments, the TLR2 moiety and/or allergen treatment disclosed herein is continued for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 18 months, at least about 24 months, at least about 3 years, at least about 5 years, or at least about 10 years.

It will be understood, that the specific dose level for any particular patient may depend upon a variety of factors including the activity of the specific compound employed, the age, body weight, general health, sex, diet, time of administration, route of administration, and rate of excretion, drug combination i.e. other drugs being used to treat the patient), and the severity of the particular disorder undergoing therapy.

The terms “treatment,” “treating,” “minimising” or “attenuating” includes the application or administration of a compound of the invention to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. These terms refer to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As described herein, there are also provided methods for desensitising and/or tolerising a subject to an allergic immune response. The subject who has received the treatment for allergy may be completely desensitised and/or tolerised, or may be continuing to receive treatment and is not yet completely desensitised and/or tolerised. It will be understood that a desensitised and/or tolerised individual will not demonstrate one or more symptoms associated with the allergy described herein or known in the art. Preferably, the desensitised and/or tolerised individual will not have any symptoms of the allergy described herein or known in the art.

A skilled person will understand that tolerisation may be achieved via the administration of allergen-specific immunotherapy in conjunction with a TLR2 moiety described herein. The regimens for subcutaneous immunotherapy vary from standard, prolonged cluster, rush or ultra-rush allergen updosing until a maintenance allergen dose that is well-tolerated by the patients is achieved. Following this is the regular administration of the maintenance dose. For instance, for subcutaneous immunotherapy the regimen includes daily or 3 times/week dosing for an initial 2-4 weeks updosing phase until the maintenance dose is reached.

In an embodiment, the method for tolerising an individual therefore includes any of the steps outlined above. In one embodiment, the subject may have a history of the particular allergy or be susceptible or at risk to the allergy as confirmed by one or more relevant clinical tests known in the art. These may include increased blood levels of allergen-specific IgE, positive skin allergy testing and/or positive allergen challenge testing by the means of oral, nasal, inhaled or conjunctival allergen exposure.

The individual may demonstrate activation of an adaptive immune response (ie Th2-driven immune response) including the presence of allergen specific IgE but does not yet have any detectable symptoms of the allergy. It will be understood that methods of preventing the development of allergy include methods of delaying the onset of the allergic symptoms in a subject.

The terms “individual,” “subject” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

### Kits

In another embodiment there is provided a kit or article of manufacture comprising any TLR2 moiety described herein and/or any allergen described herein, a pharmaceutically acceptable salt, diluent or excipient and/or pharmaceutical composition as described above. Further, the kit may comprise instructions for use in any method or use of the invention as described herein.

In other embodiments there is provided a kit for use in a therapeutic and/or prophylactic application mentioned above, the kit comprising:
a container holding a therapeutic composition in the form of a TLR2 moiety described herein and/or any allergen described herein, or a pharmaceutically acceptable salt, diluent or excipient or pharmaceutical composition;
a label or package insert with instructions for use.

In certain embodiments the kit may contain one or more further active principles or ingredients for treatment of the allergy.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat an allergy described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat or prevent an allergy described herein.

### EXAMPLES

In the studies described herein the effect of treatment with TLR2 agonist, INNA-001 or other (i.e. INNA-003 or INNA-006), on allergen-driven immune responses of immune cells isolated from blood of allergic patients has been be evaluated, in terms of proliferation, cytokine generation and induction of effector or regulatory cell phenotypes. In this type of in vitro systems, a reduction by 50% or more of allergen-driven immune responses, as indicated by the production of allergen-driven Th2 cytokines, is considered a strong indication of the compound's protective, “anti-allergic” properties.

### Example 1: Assessment of the In Vitro Effect of INNA-001 and Other Compounds on Human Allergen-Driven Th2 Cell Responses

PBMC from patients with allergy to Timothy grass pollen (Phleum pratense) were cultured for 6 days in the presence or absence of allergen and INNA-001 or control compounds. Allergen-induced proliferation and cytokine production were assessed and the phenotype of proliferating allergen-stimulated T cells was determined.

INNA-001 Suppresses Allergen-Driven Proliferation and Th2 Cytokine Production by PBMC from Allergic Donors.
A) Treatment of PBMC from allergic donors with INNA-001 resulted in substantial suppression of allergen-driven pathologic Th2 responses, as indicated by significant reduction, by more than 50% (at 3 μg/ml), of the levels of allergen-stimulated IL-4, IL-5 and IL-13 in the cell culture supernatants (FIG. 1). INNA-001 also reduced the magnitude of allergen-driven Th cell proliferation, as measured by incorporation of tritiated methyl-thymidine at day 6 (FIG. 1).

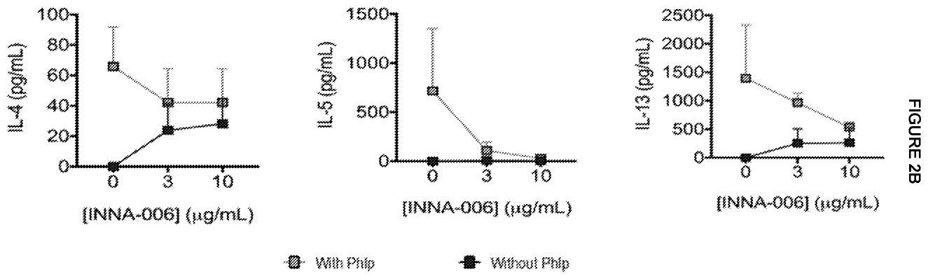

In parallel PBMC cell cultures, the effect of INNA-001 was compared to that of the control INNA compound, INNA-003. Treatment of PBMC with INNA-003 was also found capable to reduce the production of allergen-driven Th2 cytokines (FIG. 2A), but to an overall lesser magnitude than INNA-001.

In other control PBMC cell cultures, the effect of INNA-006 was assessed. Treatment of PBMC with INNA-006 was also found capable to reduce the production of allergen-driven Th2 cytokines (FIG. 2B), but to a lesser magnitude than INNA-001.

### INNA-001 Suppresses the Expansion of Allergic Disease-Related Th Cell Phenotypes in Response to Allergen Stimulation

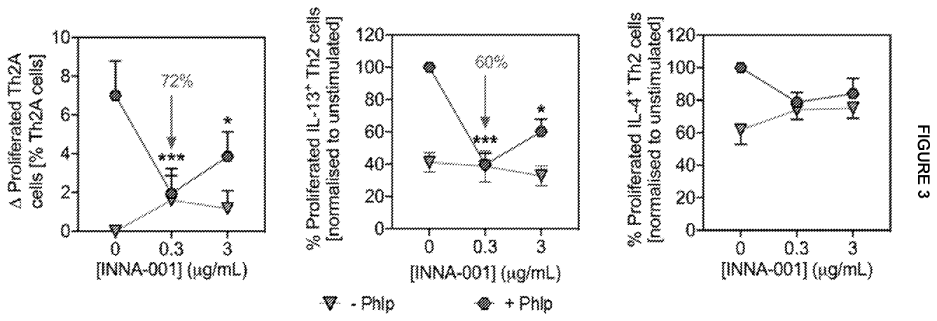

B) Allergen stimulation of PBMC from grass allergic donors treated with INNA-001 resulted in significant reduction of the expansion of Th2A (CD4⁺CRTH2⁺CD27-CD49d⁺CD161⁺) cells, as indicated by Flow Cytometry analysis of cells harvested following 6 days culture (FIG. 3). Th2A cells are terminally differentiated Th cells, with a stable phenotype, that drive allergy pathophysiology. Further analysis of the Th2 cell phenotypes indicated that treatment with INNA-001 significantly reduced the numbers of proliferating, IL-13 producing-Th2 (CD4⁺CD27⁻CRTH2⁺IL-13⁺) cells and suppressed, albeit to a lesser extent, the numbers of proliferating, IL-4 producing-Th2 (CD4⁺CD27⁻ CRTh2⁺IL-4⁺) cells (FIG. 3).

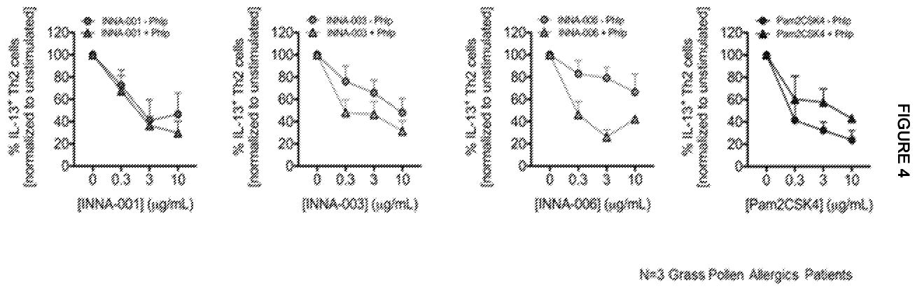

Treatment of PBMC from allergic donors with INNA-001 resulted in substantial reduction of the number of proliferating IL-13 producing-Th2 (CD4+CD27−CRTH2+IL−13+) cells in response to allergen stimulation, as indicated by Flow Cytometry analysis of cells harvested following 6 days in vitro culture in the presence or absence of allergen (FIG. 4).

Treatment of PBMC from allergic donors with INNA-003 or INNA-006 in the presence of allergen resulted in suppression of the number of proliferating IL-13 producing-Th2 (CD4+CD27−CRTH2+IL-13+) cells. The effect of treatment with these two compounds, and particularly of INNA-006, seemed to be less pronounced in the absence of allergen (FIG. 4).

Comparative treatment of the PBMC cells with Pam2CySK4 indicated that Pam2CySK4 in the presence of allergen also reduced the number of proliferating Th2 (CD4+CD27−CRTH2+IL-13+) cells, but to a lesser degree than INNA-001 (FIG. 4).

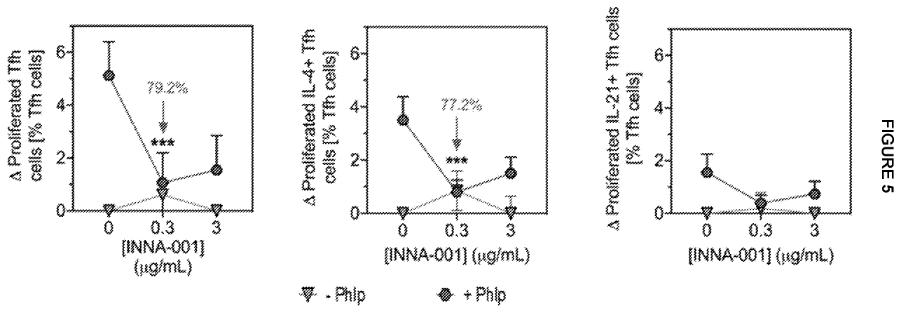

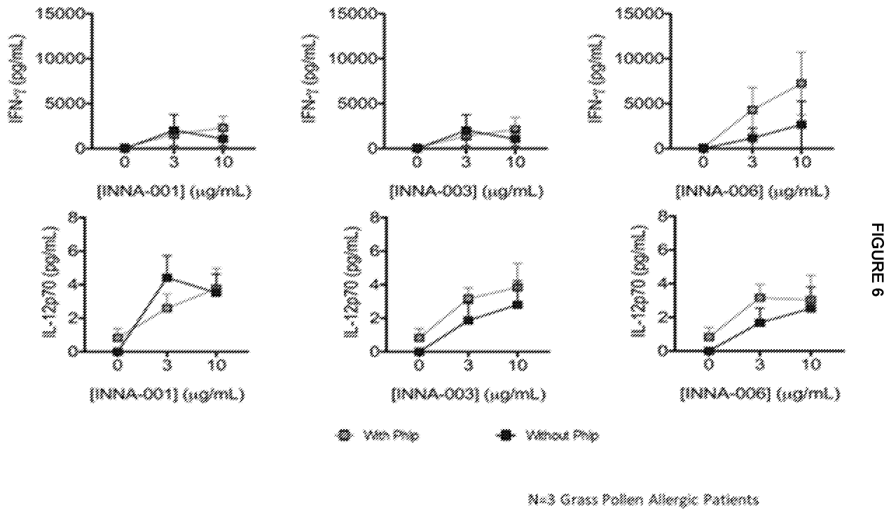

The effect of treatment with INNA-001 on the expansion of T follicular helper (Tfh) (CD4⁺CXCR5⁺PD-1⁺) cells in response to allergen stimulation was also examined. Allergen stimulation of PBMC treated with INNA-001 resulted in significant reduction of the numbers of Tfh cells, as indicated by Flow Cytometry analysis of cells harvested following 6 days culture (FIG. 5). The number of proliferating, IL-4 producing, Tfh (CD4⁺CXCR5⁺PD-1⁺IL-4⁺) cells was significantly reduced. Furthermore, a trend for a reduction in the number of proliferating, IL-21 producing, Tfh (CD4⁺CXCR5⁺PD-1⁺IL-21⁺) cells (FIG. 5) was also observed.

### Example 2: Assessment of the In Vitro the Mechanisms Mediating the Suppressive Effect of INNA-001 on Human, Allergen-Driven Th2 Responses

PBMC from patients with allergy to Timothy grass pollen (Phleum pratense) were cultured for 6 days in the presence or absence of allergen and INNA-001 or control compounds and the production of Th1 or T regulatory—type cytokines was assessed.

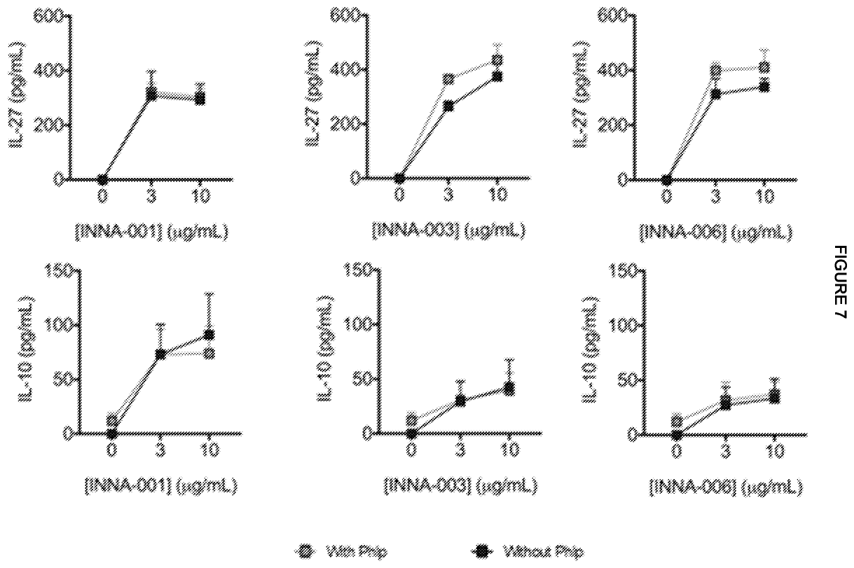

PBMC Treatment with INNA-001 does not Promote the Production of Counter-Regulatory Th1-Type Cytokines
A) Treatment of allergen-stimulated PBMC, from allergic patients, with INNA-001 did not result in enhancement of the production of Th1-type cytokines, such as IFN-gamma, IL-12p70, in the cell culture supernatants. Similarly, treatment of allergen-stimulated PBMC with INNA-003 did not affect Th1-type cytokine production, while treatment with INNA-006 slightly enhanced the generation of IFN-gamma, with no obvious effect on IL-12p70 production (FIG. 6). Treatment with all three INNA compounds induced slight increase of IL-17 A production (data not shown). The effect of treatment with INNA-001 on Th1 cytokine production did not seem to be influenced by the co-presence of allergen-stimulation. However, treatment with INNA-006 seemed to induce higher levels of IFN-gamma when there was parallel allergen stimulation, possibly indicating a differential mechanism of action from INNA-001.

### INNA-001 Induces Counter-Regulatory IL-10 Production

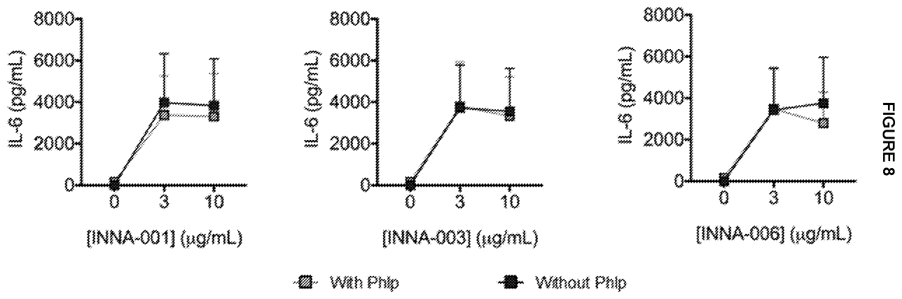

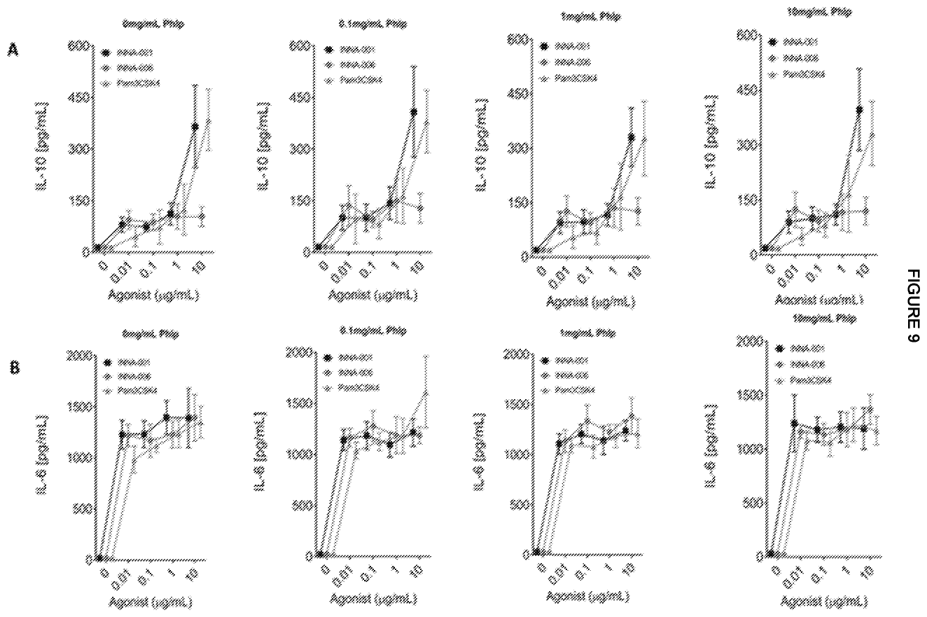

B) Treatment of PBMC from allergic patients with INNA-001, in the presence or absence of allergen, induced clear increase in IL-27 and IL-10 production in the cell culture supernatants. PBMC treatment with the control compounds INNA-003 and INNA-006 resulted in analogous increase in IL-27 production, but less pronounced enhancement of IL-10 induction (approximately 50% lower) (FIG. 7). IL-27 is involved in suppression of immune responses through upregulation of IL-10 induction. It is also considered a differentiation factor for IL-10 producing T regulatory cells.
   Treatment with INNA-001 Enhances PBMC IL-6 Production.
C) Treatment of PBMC, from grass pollen allergic patients, with INNA-001 or control compounds INNA-003 or INNA-006, in the presence or absence of allergen, resulted in marked enhancement of IL-6 production in the cell culture supernatants (FIG. 8). No overall differences in the amounts of induced IL-6 were observed between the tested compounds. Parallel allergen stimulation did not seem to influence the IL-6 upregulation.

### Example 3: Assessment of In Vitro Kinetics of INNA-001-Induced IL-10 Production by Human PBMC

PBMC from patients with allergy to Timothy grass pollen (Phleum pratense) or cat (Fel d 1) were cultured for 24 hours, 72 hours or 6 days, in the presence or absence of allergen and INNA-001, INNA-006 or Pam3CSK4 (a TLR2 agonist known to induce IL-10 production) and the production of IL-10 and IL-6 (used as a control, TLR2-induced cytokine) was measured in the cell culture supernatants.

### INNA-001, but not INNA-006, Induces Dose-Dependent Upregulation of IL-10 Production.

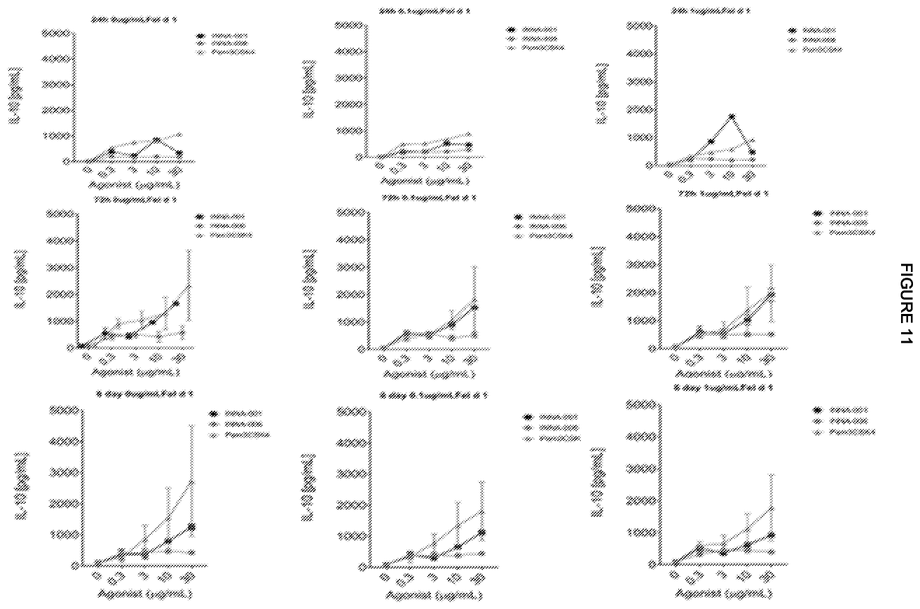

A) Treatment of PBMC from allergic donors with INNA-001 or Pam3CSK4, in the presence or absence of allergen stimulation, resulted in rapid, dose-dependent generation of IL-10, within 24 hours. The increase of IL-10 levels continued to be observed at 72 hours and remained high at 6 days of culture (FIG. 10). Similarly, treatment with Pam3CSK4 also triggered rapid, dose-dependent induction of IL-10 within 24 hours that remained high, but with declining levels, over the 6 days culture period. On the contrary, treatment with INNA-006 resulted in lower, dose-independent increase of IL-10 (FIG. 10).

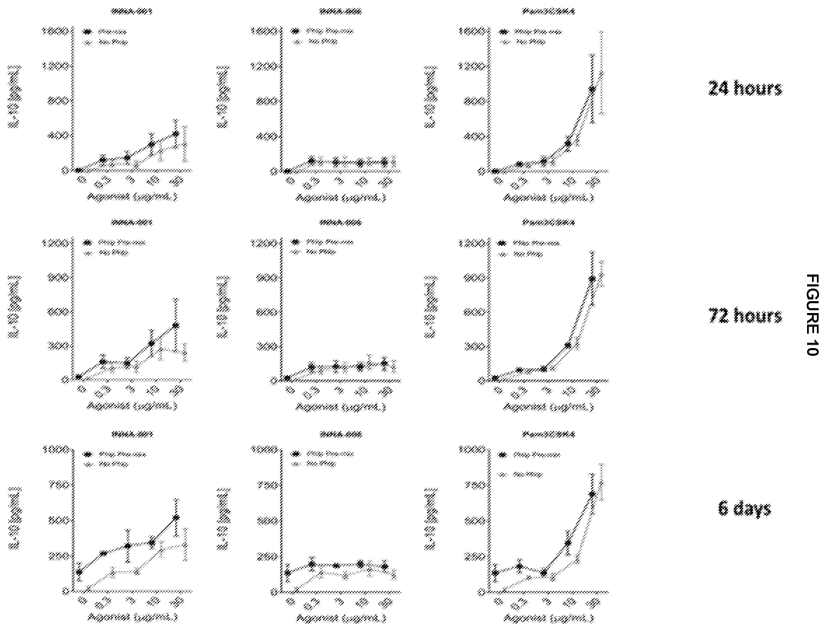

The presence of parallel allergen stimulation did not appear to considerably influence the effect of INNA-001, INNA-006 and Pam3CSK4, on IL-10 production by PBMC, particularly at the early time points.
B) To examine whether the differential effect of INNA-001, compared to INNA-006, on IL-10 production by PBMCs was also seen on other TLR2-triggered cytokines, the levels of IL-6 were measured in 24 hours cell cultures. The results showed that, in contrast to their distinctive effect on IL-10 induction (FIG. 9A), treatment with INNA-001, INNA-006 or Pam3CSK4, induced a similar pattern of strong IL-6 generation, that reached almost plateau levels following stimulation with the lowest compound concentrations tested (FIG. 9B). The presence of parallel allergen stimulation did not appear to influence the effect of INNA-001, INNA-006 or Pam3CSK4 on IL-6 production. IL-6 mediates the acute phase of the innate immune defence. IL-6 also has anti-inflammatory properties and is involved in the enhancement of counter-regulatory mechanisms in synergy with IL-10.
C) To confirm the ability of INNA-001 to drive IL-10 induction by PBMC from various types of allergic patients, a similar set of experiments was conducted using PBMC isolated from blood samples of cat allergic subjects. The findings were analogous and treatment with INNA-001 or Pam3CSK4, but not INNA-006, stimulated dose-dependent upregulation of IL-10, at 24 hours, 72 hours and 6 days of PBMC cultures in the presence of the major cat allergen Feld 1 (FIG. 11).

### Example 4: Assessment of the In Vitro Effect of INNA-001 on Induction of IL-10 by Human Blood Monocytes

The observed early increase of IL-10 induction, within 24 hours, following stimulation of PBMC with TLR2 agonists suggests that a probable IL-10 source are the monocyte populations. To examine it, PBMC from patients with allergy to Timothy grass pollen (Phleum pratense) were cultured with INNA-001 or control TLR2 agonist compounds, in the presence or absence of allergen, for 24 hours. At 24 hours the cells were harvested, washed and stained for IL-10 and the monocyte markers CD16 and CD14, using relevant anti-human antibodies. Analysis of the monocyte populations (classical and non-classical monocytes) was performed by Flow Cytometry. Lymphoid cells were excluded using antibodies against lineage markers CD3, CD4, CD15, CD19, CD56.

### INNA-001, in the Presence of Allergen, Selectively Upregulates the Induction IL-10 Producing, Non-Classical Monocytes.

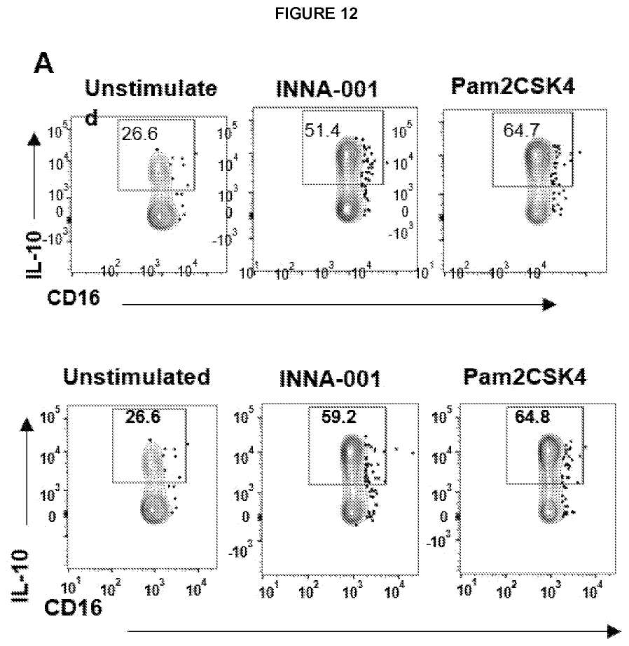

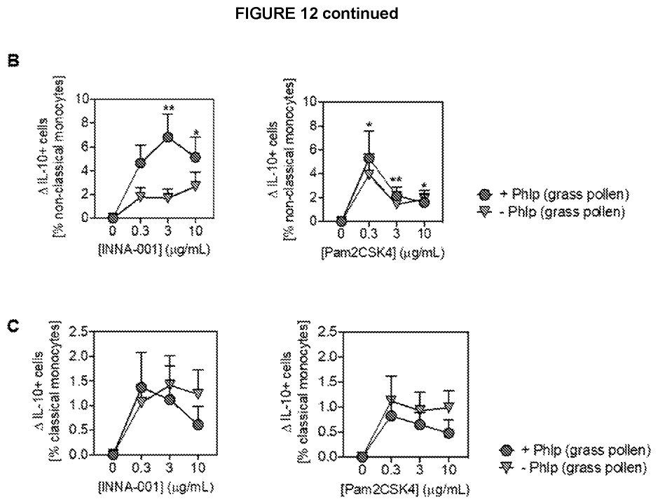

Treatment of PBMC from grass allergic patients with INNA-001 induced marked expansion of IL-10⁺, non-classical CD14^{low}/CD16⁺ monocytes (which are known to be involved in immune surveillance and homeostasis). The numbers of IL-10 producing, non-classical monocytes appeared to be significantly higher when the PBMC were cultured in the presence of grass pollen allergen (FIG. 12A and FIG. 12B).

In control experiments, treatment of PBMC with the TLR2/6 agonist Pam2CSK4 also resulted in increase, although slightly less pronounced, of the number of IL-10⁺, non-classical CD14^{low}/CD16⁺ monocytes (FIG. 12A and FIG. 12B). Interestingly however, the effect of Pam2CSK4, in contrast to INNA-001, was not influenced by the presence of allergen in the cell cultures.

Treatment of PBMC with INNA-001, as well as with Pam2CSK4, induced lower increase of the numbers of IL-10⁺, classical CD14^{high}/CD16⁻ monocytes (FIG. 12C). For both compounds this effect appeared to be independent of the presence of allergen.

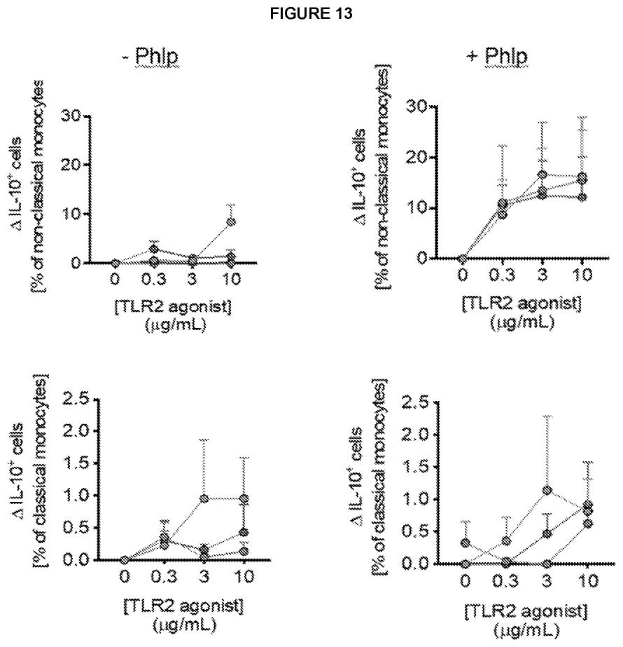

Treatment of PBMC from grass allergic patients with INNA-003 and INNA-006 also resulted in increase of the numbers of IL-10⁺, non-classical CD14^{low}/CD16⁺ monocytes (FIG. 13) and to a lesser extend of the numbers of IL-10⁺, classical CD14^{high}/CD16−monocytes (FIG. 13) The three tested INNA compounds had a similar pattern of IL-10 producing monocytes induction and the increase in the numbers of IL-10⁺, non-classical CD14^{low}/CD16⁺ monocytes was more pronounced in the presence of allergen in the cell cultures.

### Example 5: Assessment of the Potential of INNA-001 to Induce CD4⁺ T Regulatory Cells Following Treatment of PBMC of Allergic Patients

PBMC from patients with allergy to Timothy grass pollen were cultured in vitro for 6 days with i) INNA-001 or control compounds in the presence or absence of allergen (Phleum pratense), or ii) with INNA-001 or control compounds in the presence or absence of non-specific polyclonal stimulation with anti-CD3 and anti-CD28. At day 6 of culture, the cells were harvested, washed and stained for the standard CD4+T regulatory cell markers, using the relevant anti-human antibodies. Analysis was performed by Flow Cytometry.

Treatment with INNA-001 Promotes the Expansion of Naïve and Memory FOXP3+CD4+T Regulatory Cells

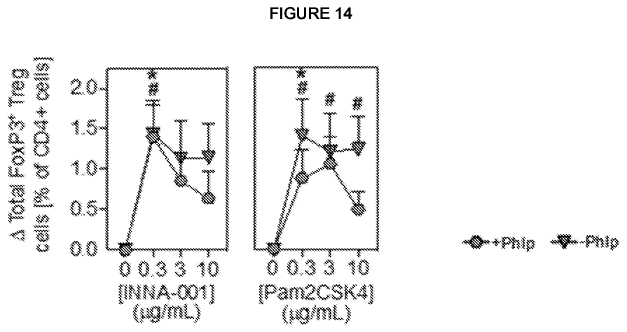

Treatment of PBMC with INNA-001 resulted in expansion of the total number of CD4⁺CD25⁺CD127⁻ regulatory T cells (FIG. 14). This effect was independent of the presence of allergen. Analogous results were observed in experiments using the control compound Pam2CSK4 (FIG. 14).

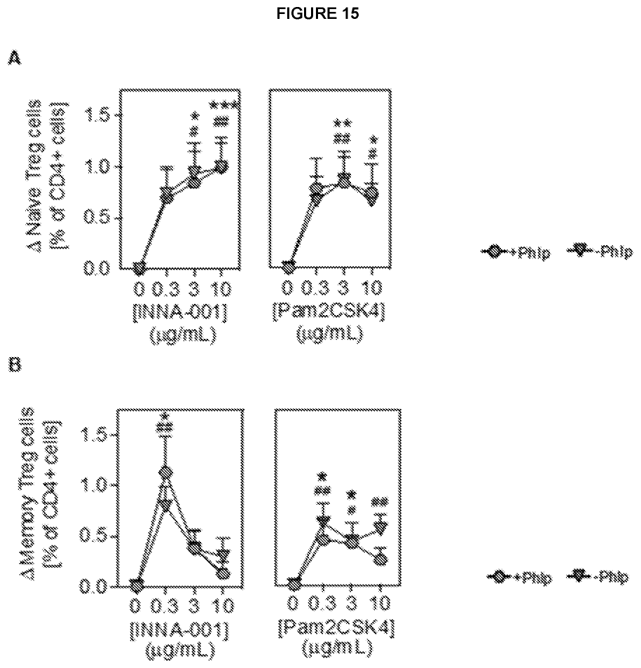

Further analysis, to identify the effect of PBMC treatment with INNA-001 on the subpopulations of naive and memory CD4⁺ T regulatory cells, revealed that the numbers of both, naive FoxP3⁺CD25⁺CD127⁻CD4⁺CD45RO⁻ cells (FIG. 15A), as well as memory FoxP3⁺CD25⁺CD127⁻CD4⁺CD45RO⁺ cells (FIG. 15B) increased. Analogous was the effect of PBMC treatment with Pam₂CSK4 on the memory and naive CD4⁺ T regulatory cell subpopulations. Interestingly, INNA-001, at the concentration of 0.3 mg/mL, induced a clearly more pronounced expansion of memory FoxP3⁺CD25⁺CD127⁻CD4⁺CD45RO⁺ cells in the presence of parallel allergen stimulation than Pam2CSK4 (FIG. 14B).

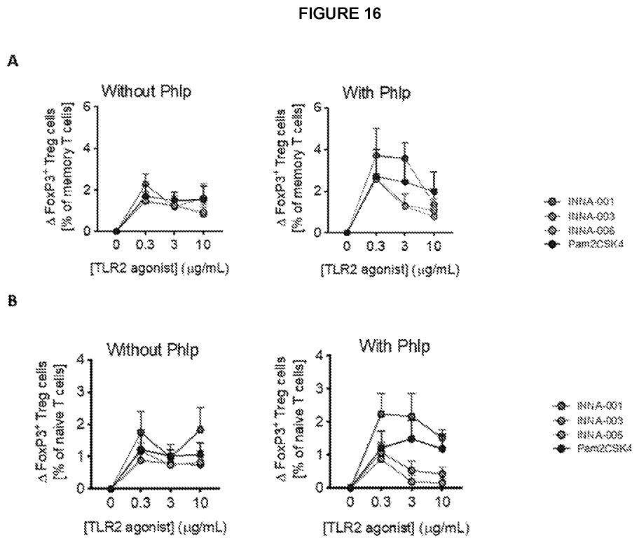

The effect of PBMC treatment with INNA-001 on the induction of Foxp3⁺ T regulatory cells was compared to that of INNA-003 and INNA-006. Both, INNA-003 and INNA-006 also promoted the induction of naïve (FIG. 16A) and memory (FIG. 16B) Foxp3⁺ T regulatory cells, in a way overall analogous to that of INNA-001. However, when allergen was also present in the cell cultures these compounds appeared to be less potent inducers of naïve and memory Foxp3⁺ T regulatory cells, than INNA-001.

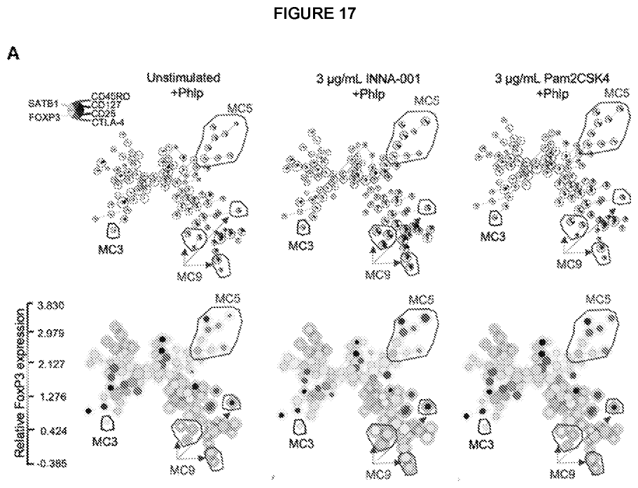

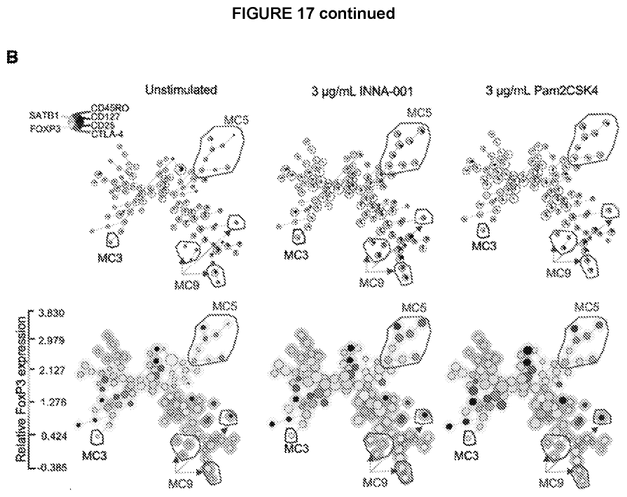

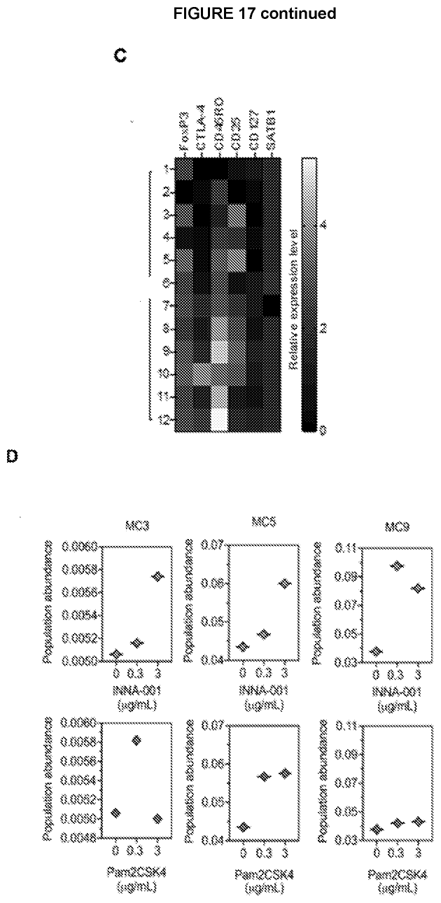

To understand the phenotype of CD4⁺ T regulatory cells induced following treatment with INNA-001 of allergen stimulated PBMC from grass allergic patients, unbiased FlowSOM analysis was performed and the expression of the T regulatory markers Foxp3, CTLA4, CD4, CD25, CD45RO, CD127 and SATB1 at single cell level was assessed. The analysis identified twelve distinct metaclusters, characterised by variable expression of the assessed cell markers. From those, three metaclusters, MC3, MC5 and MC9, were more prominent and presented higher expansion in response to INNA-001-stimulation (FIG. 17A and and FIG. 17B). In control experiments, the same metaclusters were also found prominent in response to PBMC treatment with Pam2CSK4 (and FIG. 17A). However, the cell abundance in each of these three metaclusters was different in cell cultures with INNA-001 versus Pam2CSK4, with MC9 cells (characterised by higher CD45RO expression) in particular, being far more abundant following INNA-001 stimulation (FIG. 17C).

### Example 6: Assessment of the In Vitro Potential of INNA-001 to Induce IL-10 Producing B Regulatory Cells from Blood Cells of Allergic Patients

From blood samples of Timothy grass pollen (Phleum pratense) allergic patients, CD19⁺ B cells were isolated and cultured for 72 hours with INNA-001, in the presence or absence of allergen (Phelp). At 72 hours the cells were harvested, washed and stained for a series of B regulatory cell markers and IL-10, using relevant anti-human antibodies. Analysis was performed by Flow Cytometry.

### INNA-001 Promotes Dose-Dependent Induction of IL-10 Producing B Regulatory Cells.

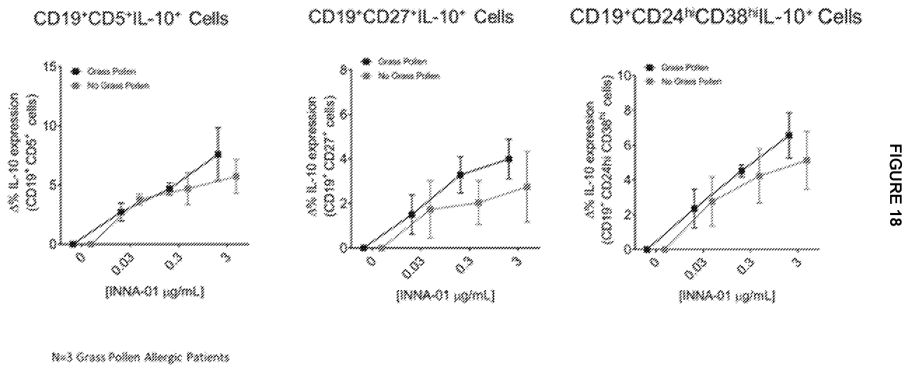

Treatment of CD19⁺ B cells, isolated from PBMC of allergic patients, with INNA-001 resulted in dose-dependent expansion of IL-10 producing B regulatory cell subsets (CD19⁺CD5⁺IL-10⁺, CD19⁺CD27⁺IL-10⁺ and CD19⁺CD24^{h}CD38^{hi}IL-10⁺) (FIG. 18). The effect of INNA-001 appeared to be independent of the presence or absence of allergen.

These data suggest that the changes observed in the INNA-001-induced B regulatory cells are different from those induced by a standard control (CpG/TLR9 agonist), known to enhance B regulatory cells expansion.

The experimental data described herein shows:
INNA-001 can effectively suppress in vitro human, allergen-driven, Th2 responses.
INNA-001 seems to mediate the suppression of allergic Th2 responses primarily through induction of counter-regulatory cytokines, such as IL-10. The effect of INNA-001 does not appear to be associated with strong induction of allergen-specific Th1 type cells, producing IFN-γ.
INNA-001 activates a series of mechanisms promoting immune tolerance and homeostasis. Specifically, INNA-001 appears to be an effective inducer of IL-10 producing monocytes, T regulatory and B regulatory cells.
The effect of INNA-001 on T regulatory cell induction in more pronounced in the presence of allergen.

1. A method of treating, attenuating or preventing an allergic immune response in a subject, comprising administering a therapeutically effective amount of a TLR2 moiety comprising, consisting essentially, or consisting of a TLR2 agonist and a polar polypeptide,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby treating, attenuating or preventing an allergic immune response in the subject.

2. A method of treating, attenuating or preventing an allergic immune response in a subject, comprising administering a therapeutically effective amount of (i) a TLR2 moiety comprising, consisting essentially, or consisting of a TLR2 agonist and a polar polypeptide; and (ii) and an allergen,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby treating, attenuating or preventing an allergic immune response in the subject.

3. Use of a TLR2 moiety comprising, consisting essentially of or consisting of a TLR2 agonist and a polar polypeptide in the preparation of a medicament for treating, attenuating or preventing an allergic immune response in a subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.

4. Use of a TLR2 moiety comprising, consisting essentially of or consisting of a (i) TLR2 agonist and a polar polypeptide; and (ii) an allergen in the preparation of a medicament for treating, attenuating or preventing an allergic immune response in a subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.

5. The method or use of any one of claims 1 to 4, wherein the lipid moiety is palmitolyl.

6. The method or use of any one of claims 1 to 4, wherein the TLR2 agonist is a lipopeptide.

7. The method or use of claim 6, wherein the lipopeptide is selected from the group consisting of Pam2Cys, Pam3Cys, Ste2Cys, Lau2Cys, and Oct2Cys.

8. The method or use of claim 7, wherein the lipopeptide is Pam2Cys.

9. The method or use of any one of claims 1 to 8, wherein polar polypeptide is a hyper-branched polypeptide.

10. The method or use of claim 9, wherein the hyper-branched polypeptide is selected from the group consisting of BE8, K8, R8 or H8.

11. The method or use of any one of claims 1 to 8, wherein polar polypeptide is a branched polypeptide.

12. The method or use of claim 11, wherein the branched polypeptide is selected from the group consisting of E4, K4, R4 or H4.

13. The method or use of any one of claims 1 to 12, wherein TLR2 agonist and the polar polypeptide are linked by at least one serine residue, preferably two serine residues.

14. The method or use of any one of claims 1 to 8, wherein the TLR2 moiety comprising, consisting essentially, or consisting of a TLR2 agonist and a polar polypeptide is Pam2CysR4.

15. A method of treating, attenuating or preventing an allergic immune response in a subject comprising administering a therapeutically effective amount of Pam2CysR4 to the subject, thereby treating, attenuating or preventing the allergic immune response in the subject.

16. A method of treating, attenuating or preventing an allergic immune response in a subject comprising administering a therapeutically effective amount of (i) Pam2CysR4; and (ii) an allergen to the subject, thereby treating, attenuating or preventing the allergic immune response in the subject.

17. Use of Pam2CysR4 in the preparation of a medicament for treating, attenuating or preventing an allergic immune response in a subject.

18. Use of (i) Pam2CysR4 and (ii) and allergen in the preparation of a medicament for treating, attenuating or preventing an allergic immune response in a subject.

19. The method or use of any one of claims 1 to 18, wherein the allergic immune response is associated with an allergy selected from the group consisting of microbe allergy; pollen allergy; aeroallergy, food allergy, animal allergy; mite or insect allergy; mould allergy; plant allergy; drug allergy; and hymenoptera venom allergy.

20. The method or use of any one of claims 1 to 18, wherein the allergen is associated with an allergy selected from the group consisting of microbe allergy; pollen allergy; animal allergy; food allergy; mite or insect allergy; mould allergy; plant allergy; drug allergy; and hymenoptera venom allergy.

21. The method or use claim 20, wherein the allergen is Timothy grass pollen allergen (Phleum pratense).

22. The method or use claim 20, wherein the allergen is the cat allergen Fel d 1.

23. The method or use of any one of claims 1 to 22, wherein the subject has one or more symptoms associated with the allergy.

24. The method or use of any one of claims 1 to 23, wherein the subject has received or is receiving a treatment in the form of an allergen.

25. The method or use of claim 21, wherein the allergen is not associated with rhinovirus-mediated exacerbation of asthma or is not associated with rhinovirus.

26. A method of desensitising and/or tolerising a subject to an allergen comprising administering a therapeutically effective amount of a TLR2 moiety comprising a TLR2 agonist and a polar polypeptide,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby desensitising and/or tolerising the subject to the allergen.

27. The method of claim 26, further comprising administering a therapeutically effective amount of an allergen.

28. The method of claim 26 or 27, further comprising identifying a subject that is allergic to an allergen.

29. The method of any one of claims 26 to 28, wherein the TLR2 moiety is Pam2CysR4.

30. A composition comprising a TLR2 moiety and an allergen
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.

31. The composition of claim 30, wherein the TLR2 moiety comprising a TLR2 agonist and a polar polypeptide and/or (ii) allergen are administered in a composition.

32. The composition of claim 30 or 31, wherein the composition comprises a pharmaceutically acceptable carrier, diluent or excipient.

33. The composition of any one of claims 30 to 32, wherein the composition is formulated for systemic, intravenous, intralymphatic, intramuscular, subcutaneous, intradermal, oral, topical, intranasal, aerozolised, epidermal or sublingual administration to the subject.

34. The composition of claim 33, wherein the composition is formulated for oral or sublingual administration to the subject.

35. A method of increasing levels of regulatory T and/or regulatory B cells in a subject, comprising administering a therapeutically effective amount of a TLR2 moiety comprising a TLR2 agonist and a polar polypeptide to the subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby increasing levels of regulatory T and/or regulatory B cells in the subject.

36. A method of increasing levels of non-classical monocytes in a subject comprising administering a therapeutically effective amount of a TLR2 moiety comprising a TLR2 agonist and a polar polypeptide to the subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby increasing levels of non-classical monocytes in the subject.

37. A method of treating, attenuating or preventing anaphylaxis in a subject comprising administering a therapeutically effective amount of a TLR2 moiety comprising a TLR2 agonist and a polar polypeptide to the subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby treating, attenuating or preventing anaphylaxis in the subject.

38. A method of increasing survival in a subject comprising administering a therapeutically effective amount of a TLR2 moiety comprising a TLR2 agonist and a polar polypeptide to the subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby increasing survival in the subject.

39. A method of treating, alleviating or preventing a Th2 immune response in a subject comprising administering a therapeutically effective amount of a TLR2 moiety comprising a TLR2 agonist and a polar polypeptide to the subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby treating, alleviating or preventing a Th2 immune response in the subject.

40. The method of any one of claims 1 to 39, wherein the administration of the TLR2 moiety is not primarily associated with a Th1 immune response.

41. The method of any one of claims 1 to 40, wherein the administration of the TLR2 moiety comprising a TLR2 agonist and a polar polypeptide is not associated with significant changes to the levels of interferon gamma (IFN gamma).

42. The method of claim 39, wherein the treatment, alleviation or prevention of a Th2 immune response is determined by assessment of levels of IL-4, IL-5 and/or IL-13.

43. A method of increasing levels of IL-10 in a subject comprising administering a therapeutically effective amount of a TLR2 moiety comprising a TLR2 agonist and a polar polypeptide to the subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof,
thereby increasing levels of IL-10 in the subject.

44. The method of any one of claims 35 to 43, wherein the TLR2 moiety is Pam2CysR4.

45. The method of any one of claims 35 to 44, further comprising administration of an allergen.

46. The method or use of any one of claims 2, 4, 16, 18, 27, 31 or 45, wherein the (i) TLR2 moiety; and (ii) allergen are administered at the same time.

47. The method of any one of claims 2, 4, 16, 18, 27, 31 or 45, wherein the (i) TLR2 moiety and (ii) allergen are administered more than once to the subject.

48. The method of any one of claims 2, 4, 16, 18, 27, 31 or 45, wherein the (i) TLR2 moiety and (ii) allergen are administered to the subject for a period of between 3 to 6 months or between 6 months and 3 years.

49. A TLR2 moiety comprising, consisting essentially of a TLR2 agonist and a polar polypeptide for treating, attenuating or preventing an allergic immune response in a subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.

50. A (i) TLR2 moiety comprising, consisting essentially of a TLR2 agonist and a polar polypeptide; and (ii) an allergen for treating, attenuating or preventing an allergic immune response in a subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.

51. A TLR2 moiety comprising, consisting essentially of or consisting of a TLR2 agonist and a polar polypeptide for use in treating, attenuating or preventing an allergic immune response in a subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.

52. A (i) TLR2 moiety comprising, consisting essentially of a TLR2 agonist and a polar polypeptide; and (ii) an allergen for use in treating, attenuating or preventing an allergic immune response in a subject,
wherein the TLR2 agonist comprises, consists essentially of or consists of a lipopeptide or lipid moiety,
wherein the lipid moiety is selected from the group consisting of palmitolyl, myristroyl, steareoyl, lauroyl, decanoyl and combinations thereof.
